(19) United States
(12) Patent Application Publication
(10) Pub. No.: US 2024/0132461 A1
(43) Pub. Date: Apr. 25, 2024

(54) FLAVONE DEAZA SPERMIDINE ANALOGUES AND THEIR USE TREATING CANCER

(71) Applicant: FLORATEK PHARMA S.A., Aubonne (CH)

(72) Inventor: Dan Florin Stoicescu, Aubonne (CH)

(21) Appl. No.: 18/275,847

(22) PCT Filed: Feb. 8, 2022
(86) PCT No.: PCT/EP2022/053042

§ 371 Date: Aug. 4, 2023

(30) Foreign Application Priority Data
Feb. 8, 2021 (GB) 2101728.0

(51) Int. Cl. C07D 311/30; A61P 35/00; C07D 405/10; C07D 405/12; C07D 413/10; C07D 493/14

(52) U.S. Cl. CPC C07D 311/30; A61P 35/00; C07D 405/10; C07D 405/12; C07D 413/10; C07D 493/14

(57) ABSTRACT

The present invention relates to chromen-4-one derivatives, and to associated multi-salts, solvates, and pharmaceutical compositions. The present invention also relates to the use of such compounds and compositions in the treatment and prevention of cancer.

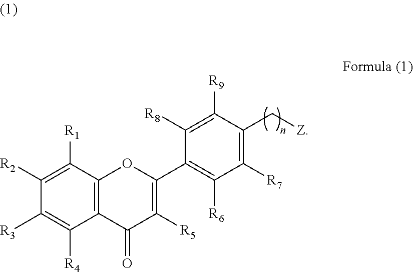

### FIELD OF THE INVENTION

The invention relates to compounds, pharmaceutical compositions comprising the same, and methods of treatment employing the same. In particular, the compounds are useful for the treatment or prevention of cancer.

### BACKGROUND

Dual-targeting or multi-targeting of malignant pathways by a single drug molecule represents an efficient, logical and alternative approach to drug combinations. A new generation of dual or multi-targeting drugs is emerging, where a single chemical entity can act on multiple molecular targets [Raghavendra N M, et al. Dual or multi-targeting inhibitors: The next generation anticancer agents. Eur J Med Chem. 2018 Jan 1;143:1277-1300]. The present invention uses a rational, bioinformatics and poly-pharmacological approach to design multi-target anticancer agents by combining flavonoid-like structures with a variety of polyamine chains from the spermidine class. Both broad molecule structures have been shown to target multiple cellular pathways leading to cancer inhibition [Kikuchi H, Yuan B, Hu X, Okazaki M. Chemopreventive and anticancer activity of flavonoids and its possibility for clinical use by combining with conventional chemotherapeutic agents. Am J Cancer Res. 2019;9(8):1517-1535; Carl W. Porter, Raymond J. Bergeron and Neal J. Stolowich. Biological Properties of N⁴-Spermidine Derivatives and Their Potential in Anticancer Chemotherapy. Cancer Res. 1982 (42) (10) 4072-4078].

The role of flavonoids as potential cancer therapies includes the inhibition of activation of pro-carcinogens, inhibition of proliferation of cancer cells, selective death of cancer cells by apoptosis, inhibition of metastasis and angiogenesis, activation of immune response against cancer cells, modulation of the inflammatory cascade and the modulation of drug resistance [Kikuchi H, Yuan B, Hu X, Okazaki M. Chemopreventive and anticancer activity of flavonoids and its possibility for clinical use by combining with conventional chemotherapeutic agents. Am J Cancer Res. 2019;9(8)1517-1535]. In spite of their promising potential in controlling the malignant process, natural flavonoids present major limitations to their clinical use due to low bioavailability and their perceived lack of specificity. Such versatile biological activity implies a great underlying complexity in the true mechanisms of action of different flavonoids, often dependent on a fine balance between pro- and anti-oxidant properties or between other beneficial and detrimental effects [Martinez Perez, et al. 2014. Novel flavonoids as anti-cancer agents: mechanisms of action and promise for their potential application in breast cancer. Biochemical Society Transactions, vol. 42, no. 4, pp. 1017 - 1023.]. As such, a systematic study of the structure activity relationship of the flavonoid structures is necessary and can be addressed by the rationale design of a series of molecules. The present invention addresses some of the limitations of flavonoid derivatives by employing a novel approach of combining such moieties with a synthetic polyamine chain with multiple potential advantages: (i) multi-targeting cancer pathways (ii) inhibition of the natural polyamine pathway which is often dysregulated in cancer and (iii) facilitating transport through the cell membrane and targeting to specific intracellular structures.

It is well-known that polyamines interact with aspartate, glutamate, and aromatic residues of a given receptor and/or enzyme mainly through the formation of ion bonds, since at physiological pH, protonation of amino groups is nearly complete. From this, the hypothesis arises that a polyamine may be a universal template able to recognize different receptor systems. This hypothesis suggests that both affinity and selectivity may be fine-tuned by inserting appropriate substituents onto the amine functions and by varying the methylene chain lengths between them on the polyamine backbone [Minarini, A. Milelli, A., Tumiatti, V. et al. Synthetic polyamines: an overview of their multiple biological activities. Amino Acids 38, 383-392 (2010).]

Polyamine metabolism is often dysregulated in cancers. In addition, the polyamine pathway is a downstream target for many oncogenes [Shantz L M, Levin V A. Regulation of ornithine decarboxylase during oncogenic transformation: mechanisms and therapeutic potential. Amino Acids. 2007;33(2):213-23]. Polyamine biosynthesis is activated in tumors, and these metabolites are important for developmental and compensatory growth in response to systemic stimuli like hormones (growth hormones, corticosteroids, androgens, and estrogens). As a result, various strategies targeting polyamine biosynthetic enzymes have been brought to the preclinical and clinical arena [Arruabarrena-Aristorena A, et al. Oil for the cancer engine: The cross-talk between oncogenic signaling and polyamine metabolism. Sci Adv. 2018;4M:1-11].

The most successful and widely used inhibitor of polyamine biosynthesis is 2-difluoromethylornithine (DFMO). DFMO was specifically designed to be an enzyme-activated irreversible inhibitor of ODC [Metcalf B W, et al. Catalytic irreversible inhibition of mammalian ornithine decarboxylase (E.C4.1.1.17) by substrate and product analogues. J. Am. Chem. Soc. 1978;100:2551-2553.]

These encouraging results in selective cancers, both in vitro and in vivo led to clinical trials with DFMO as a single agent. Although DFMO was exceedingly well tolerated, there were no significant clinical responses observed in the early trials. More recently, a resurgence of interest in DFMO as a single agent has occurred in the treatment of neuroblastoma [Bassiri H, et al. Translational development of difluoromethylornithine (DFMO) for the treatment of neuroblastoma. Transl. Pediatr. 2015;4:226-238] and as a chemoprevention agent, alone or in various combinations [Gerner E W, Meyskens F L Jr. Polyamines and cancer: old molecules, new understanding. Nat Rev Cancer. 2004 Oct;4(10):781-92].

Another rational for linking polyamines to bioactive moieties is not only to use the polyamine transport system to enter the cell, but also to direct the agent to its intracellular molecular target, which can be mitochondria or other anionic structures [Murray-Stewart T R, et al. Targeting polyamine metabolism for cancer therapy and prevention. Biochem J. 2016;473(19) 2937-2953].

### SUMMARY OF THE INVENTION

A first aspect of the invention provides a compound of formula (1):

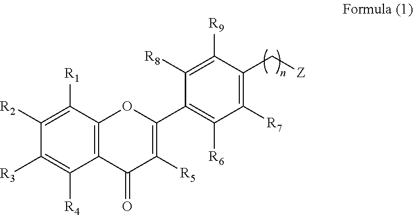

wherein:
Z is selected from: —NR¹¹R¹²;
   —N(R¹⁰)—(CH₂)ₚ—NR¹¹R¹²; and
   —N(R¹⁰)—(CH₂)_{q}—N(R¹⁰)—(CH₂)_{q}—NR¹¹R¹²;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-6;
each p is independently an integer selected from 1 to 4; and
each q is independently an integer selected from 1 to 4.

In one embodiment, the compound may be a compound of Formula (1A):

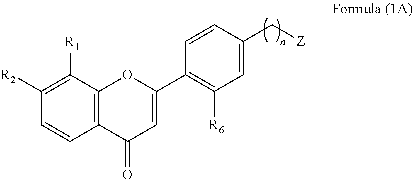

wherein R¹, R², R⁶, n and Z are as defined herein.

In one embodiment, the compound may be a compound of formula (2):

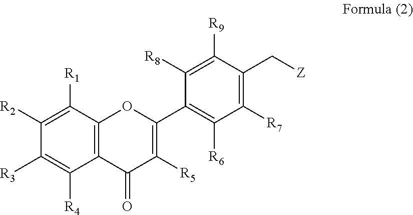

wherein R¹-R⁹ and Z are as defined herein.

A second aspect of the invention provides a compound selected from the group shown in Table A herein.

A third aspect of the invention provides pharmaceutically acceptable salt, multi-salt, solvate or prodrug of the compound of the first or second aspect of the invention.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F —CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH ₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)) A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

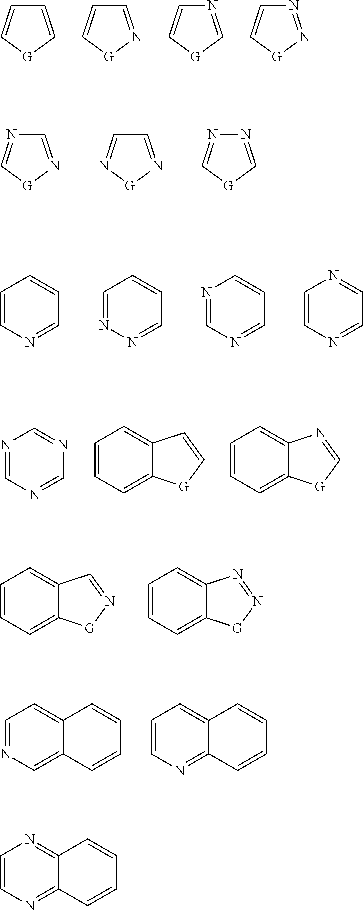

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})—or —R^{α}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

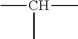

is replaced by

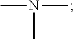

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

### DESCRIPTION OF FIGURES

FIG. 1 shows IC50 curve of SND164 (Cpd G) activity against brain carcinoma.

FIG. 2 shows IC50 curve of SND164 (Cpd G) activity against breast carcinoma.

FIG. 3 shows IC50 curve of SND164 (Cpd G)activity against colon carcinoma.

FIG. 4 shows IC50 curve of SND164 (Cpd G) activity against chronic myelogenous leukemia (CML).

FIG. 5 shows IC50 curve of SND164 (Cpd G) activity against small cell lung carcinoma PDX.

### DETAILLED DESCRIPTION OF THE INVENTION

A first aspect of the invention provides a compound of formula (1):

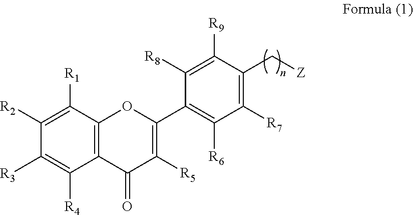

wherein:
Z is selected from: —NR¹¹R¹²;
   —N(R¹⁰)—(CH₂)ₚ—NR¹¹R¹²;
   —N(R¹⁰)—(CH₂)_{q}—N(R¹⁰)—(CH₂)_{q}—NR¹¹R¹²; and
R¹ and R², independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 -R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl, or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-6;
each p is independently an integer selected from 1 to 4; and
each q is independently an integer selected from 1 to 4.

R¹ and R², independently, are selected from —OH, —O—C₁₋₄ alkyl, —OC(O)R₁₃, —OC(O)NHR¹³, —OC(O)N(R¹³)₂. In one embodiment, R¹ and R², independently, are selected from —OH, and —O—C₁₋₄ alkyl. In one embodiment, R¹ and R², independently, are selected from —OH and —OCH₃.

In one embodiment, R¹ and R² together form —O—CH₂—O—.

R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. In one embodiment, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}. In one embodiment, R³, R⁴, R⁵, R⁶, R⁷, R⁸, and R⁹, independently, are selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}. In one embodiment, R³, R⁴, R⁵, R⁶, R⁷, R⁸, and R⁹, independently, are selected from H; halo; —CN; —NO₂; —RP; —NH₂; —NHR^{β}; —N(R^{β})₂; —CHO; —COR^{β}; —COOH; —COOR^{β}; and —OCOR^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}. In one embodiment, R³, R⁴, R⁵, R⁶, R⁷, R⁸, and R⁹, independently, are selected from H; halo; —CN; —NO₂; and —NH₂. In one embodiment, R³, R⁴, R⁵, R⁶, R⁷, R⁸, and R⁹, are H.

In one embodiment, R¹ and R², independently, are selected from —OH, —O—C₁₋₄ alkyl, —OC(O)R₁₃, —OC(O)NHR¹³, —OC(O)N(R¹³)₂; and R³-R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹ and R², independently, are selected from —OH, and —O—C₁₋₄ alkyl. For example, R³-R⁹ are independently selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —O—C₁₋₄ alkyl. For example, R³-R⁹ are independently selected from H; halo; —CN; -NO2; -RP; -NH 2 ; -NHRP; -N(RP)2; -CHO; -CORP; -COOH; -COORP; and -OCORP.

In one embodiment, R¹ and R 2 , independently, are selected from —OH, and -OCH 3 . For example, R3 - R9, are independently selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —OCH₃. For example, R³-R⁹, are independently selected from H; halo; —CN; —NO₂; and —NH₂.

In one embodiment, R¹ and R², independently, are selected from —OH, and —OCH₃. For example, R³-R⁹, are H.

In one embodiment, R¹ and R² are independently selected from —OH, —O—C₁₋₄ alkyl, —OC(O)R₁₃, —OC(O)NHR¹³, —OC(O)N(R¹³)₂; R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R² are independently selected from —OH and —O—C₁₋₄ alkyl, e.g. —OH and —OCH₃; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; 13 N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R² are independently selected from —OH and —O—C₁₋₄ alkyl; and R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ , and R², independently, are selected from —OH and —OCH₃; and R³, R⁴, R⁵, R⁶, R⁷, R⁸, and R⁹, independently, are selected from H; halo; —CN; —NO₂; —SH; —SO₂H; and —NH₂. For example, R³, R⁴, R⁵, R⁶, R⁷, R⁸, and R⁹ are H.

In one embodiment, R¹ is —O—C₁₋₄ alkyl, e.g. —O—Me; R² is OH; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; 13 SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R 2 are OH; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, Z is —NR¹¹R¹².

In one embodiment, Z is —N(R¹⁰)—(CH₂)ₚ—NR¹¹R¹².

In one embodiment, Z is —N(R¹⁰)—(CH₂)_{q}—N(R¹⁰)—(CH₂)_{q}—NR¹¹R¹².

Each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β}.

For example, each R¹⁰ may independently be selected from H, C₁₋₆ alkyl, and C₂-C₄ alkenyl.

For example, each R¹⁰ may independently be selected from H, C₁₋₃ alkyl, and C₂-C₄ alkenyl.

For example, each R¹⁰ is independently selected from H and C₁₋₆ alkyl.

For example, each R¹⁰ may independently be selected from H and C₁₋₃ alkyl.

For example, each R¹⁰ may independently be selected from H and —CH₃.

R¹¹ and R¹² are independently selected from H, C₁₋₆-alkyl (e.g. methyl or ethyl), C₂-C₆ alkenyl, C₂₋₆ alkynyl, C₃₋₁₀ cycloalkyl (e.g. adamantyl), and benzyl, wherein each R¹¹ and R¹², when not H, are independently optionally substituted with 1 or 2 —R^{↑}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl (e.g. methyl or ethyl), C₃₋₁₀ cycloalkyl (e.g. adamantyl), and benzyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

For example, R¹¹ and R¹² are independently selected from H and C₁₋₆ alkyl (e.g. methyl or ethyl), C₃₋₁₀ cycloalkyl (e.g. adamantyl), and benzyl; wherein each R¹¹ and R¹², when not H, are independently optionally substituted with 1 or 2 —R^{β}.

For example, R¹¹ is H, and R¹² is selected from H and C₁₋₆ alkyl (methyl or ethyl), C₃₋₁₀ cycloalkyl (e.g. adamantyl), and benzyl; wherein R¹², when not H, is optionally substituted with 1 —R^{β}.

For example, R¹¹ and R¹² are both H.

For example, R¹¹ is H, and R¹² is benzyl, optionally substituted with 1 —R^{β}. For example, R¹¹ is H, and R¹² is benzyl substituted with methoxy, e.g. R¹² is ortho-methoxy-benzyl. When R¹¹ and R¹² together form a 5- or 6-membered heterocycle as described above, it may be a 5- or 6-membered heterocycle optionally having one additional heteroatom selected from N and O; wherein the 5- or 6-membered heterocycle is optionally substituted with 1 or 2 C₁₋₄ alkyl or benzyl. In this respect, the 5- or 6-membered heterocycle may be morpholine, piperidine, piperazine, or pyrrolidine optionally substituted with 1 or 2 C₁₋₄ alkyl or benzyl. For example, the 5- or 6-membered heterocycle may be morpholine, piperazine, 4-methyl piperazine, or pyrrolidine.

In one embodiment, n is an integer from 1 to 4.

For example, n may be 3 or 4.

For example, n may be 1.

Each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁- C₄ haloalkyl), —O(C₃-C₇ cycloalkyl), halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl) and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl) and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₂ alkyl and —O(C₁₋₂ alkyl), and any —R^{β} may optionally be substituted with one or more halo, —OH, —NH₂, —CN, or —NO₂ groups.

Each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

For example, each R¹³ is independently selected from a H and C₁-C₃ alkyl, wherein any —R¹³ may optionally be substituted with one or more —R¹⁴.

For example, each R¹³ is independently selected from a H and C₁-C₂ alkyl.

Each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

For example, each R¹⁴ is independently selected from a C₁-C₆ alkyl, halo, —NO₂, —CN, —OH, —NH₂, mercapto, formyl, carboxy, carbamoyl, and C₁₋₆ alkoxy, wherein any —R₁₄ may optionally be substituted with one or more —R₁₅.

Each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

For example, each —R¹⁵ is independently selected from halogen, nitro, cyano, hydroxy, trifluoromethoxy, trifluoromethyl, amino, formyl, and carboxy.

Each p is independently an integer selected from 1 to 4.

For example, each p is independently an integer selected from 2 to 4.

For example, each p is independently selected from 3 and 4.

Each q is independently an integer selected from 1 to 4.

For example, each q is independently an integer selected from 2 to 4.

For example, each q is independently selected from 3 and 4.

For example, Z may be —N(R¹⁰)—(CH₂)₃—N(R¹⁰)—(CH₂)₄—NR¹¹R¹².

For example, Z may be —N(R¹⁰)—(CH₂)₄—N(R¹⁰)—(CH₂)₃—NR¹¹R¹².

In one embodiment, Z is —NR¹¹R¹². For example, Z is —NR¹¹R¹²; R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —NR¹¹R¹² and n is 3 or 4.

In one embodiment, Z is —N(R¹⁰)—(CH₂)ₚ—NR¹¹R¹². For example, Z is —N(R¹⁰)—(CH₂)ₚ—NR¹¹R¹²; R¹⁰ is H or C₁₋₆ alkyl; and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —N(R¹⁰)—(CH₂)ₚ—NR¹¹R¹²; p is 1-4; and n is 1-6. For example, p is 2-4, for example 2 or 3; and n is 2-5, for example 3 or 4.

In one embodiment, Z is —N(R¹⁰)—(CH₂)_{q}—N(R¹⁰)—(CH₂)_{q}—NR¹¹R¹²; and q is independently selected from 1-4. For example, q may be 2, 3 or 4. For example, Z is —N(R¹⁰)—(CH₂)_{q}—N(R¹⁰)—(CH₂)_{q}—NR¹¹R¹²; each R¹⁰ is independently selected from H and C₁₋₆ alkyl; and and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, R³, R⁴, R⁵, R⁷, R⁸ and R⁹ are H, for example as represented by Formula 1A below.

In one embodiment, the compound may be a compound of Formula 1A:

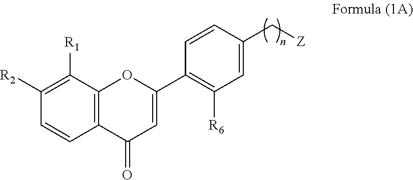

wherein R¹, R², R⁶, n and Z are as defined herein.

For example, the compound may be a compound of Formula (1A) wherein:
Z is selected from: —NR¹¹R¹²;
   —N(R¹⁰)—(CH₂)ₚ—NR¹¹R¹²; and
   —N(R¹⁰)—(CH₂)_{q}—N(R¹⁰)—(CH₂)_{q}—NR¹¹R¹²;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R⁶ is selected from H; halo; —CN; —NO₂; —R^{β}; —OH, —OR^{β}; —SH; —SR^{β}; —SOR^{β}; —SO₂H; —SO₂R⁶² ; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl, or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-6;
each p is independently an integer selected from 1 to 4; and
each q is independently an integer selected from 1 to 4.

R¹ and R², independently, are selected from —OH, —O—C₁₋₄ alkyl, —OC(O)R₁₃, —OC(O)NHR¹³, —OC(O)N(R¹³)₂. In one embodiment, R¹ and R², independently, are selected from —OH, and —O—C₁₋₄ alkyl. In one embodiment, R¹ and R², independently, are selected from —OH and —OCH₃.

In one embodiment, R¹ and R² together form —O—CH₂—O—.

R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. In one embodiment, R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}. In one embodiment, R⁶ is selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}. In one embodiment, R⁶ is selected from H; halo; —CN; —NO₂; —R^{β}; —NH₂; —NHR^{β}; —N(R^{β})₂; —CHO; —COR^{β}; —COOH; —COOR^{β}; and —OCOR^{β}. In one embodiment, R⁶ is selected from H; halo; —CN; —NO₂; —R^{β}; —NH₂; —NHR^{β}; —N(R^{β})₂; —CHO; —COR^{β}; —COOH; and —COOR^{β}. In one embodiment, R⁶ is selected from H; halo; —CN; —NO₂; and —NH₂. In one embodiment, R⁶ is H.

In one embodiment, R¹ and R², independently, are selected from —OH, —O—C₁₋₄ alkyl, —OC(O)R₁₃, —OC(O)NHR¹³, —OC(O)N(R¹³)₂; and R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹ and R², independently, are selected from —OH, and —O—C₁₋₄ alkyl. For example, R⁶ is selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —O—C₁₋₄ alkyl. For example, R⁶ is selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —OCH₃. For example, R⁶ is selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —OCH₃. For example, R⁶ is selected from H; halo; —CN; —NO₂; and —NH₂.

In one embodiment, R¹ and R², independently, are selected from —OH, and —OCH₃. For example, R⁶ is H.

In one embodiment, R¹ and R² are independently selected from —OH, —O—C₁₋₄ alkyl, —OC(O)R₁₃, —OC(O)NHR¹³, —OC(O)N(R¹³)₂; R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H; halo; —CN; —NO₂; —SH; —SO₂H; —NH₂; —CHO; —COOH. For example, R⁶ is H.

In one embodiment, R¹ and R² are independently selected from —OH and —O—C₁₋₄ alkyl, e.g. —OH and —OCH₃; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H; halo; —CN; —NO₂; —SH; —SO₂H; —NH₂; —CHO; —COOH. For example, R⁶ is H.

In one embodiment, R¹ and R² are independently selected from —OH and —O—C₁₋₄ alkyl; and R⁶ is selected from H; halo; —CN; —NO₂; —SH; —SO₂H; —NH₂; —CHO; —COOH. For example, R⁶ is H.

In one embodiment, R¹, and R², independently, are selected from —OH and —OCH₃; and R⁶ is selected from H; halo; —CN; —NO₂; —SH; —SO₂H; and —NH₂. For example, R⁶ is H.

In one embodiment, R¹ is —O—C₁₋₄ alkyl, e.g. —O—Me; R² is OH; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H; halo; —CN; —NO₂; —SH; —SO₂H; —NH₂; —CHO; —COOH. For example, R⁶ is H.

In one embodiment, R¹ and R² are OH; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H; halo; —CN; —NO₂; —SH; —SO₂H; —NH₂; —CHO; —COOH. For example, R⁶ is H.

In one embodiment, Z is —NR¹¹R¹².

In one embodiment, Z is —N(R¹⁰)—(CH₂)ₚ—NR¹¹R¹².

In one embodiment, Z is —N(R¹⁰)—(CH₂)_{q}—N(R¹⁰)—(CH₂)_{q}—NR¹¹R¹².

Each R¹⁰ is independently selected from H, C₁₋₆ alkyl, C₂-C₆ alkenyl, C₂₋₆ alkylnyl, C₃₋₁₀ cycloalkyl, and benzyl, wherein each R¹⁰, when not H, is independently optionally substituted with 1 or 2 —R^{β}.

For example, each R¹⁰ may independently be selected from H, C₁₋₆ alkyl, and C₂-C₄ alkenyl.

For example, each R¹⁰ may independently be selected from H, C₁₋₃ alkyl, and C₂-C₄ alkenyl.

For example, each R¹⁰ is independently selected from H and C₁₋₆ alkyl.

For example, each R¹⁰ may independently be selected from H and C₁₋₃ alkyl.

For example, each R¹⁰ may independently be selected from H and —CH₃. R¹¹ and R¹² are independently selected from H, C₁₋₆-alkyl (e.g. methyl or ethyl), C₂-C₆ alkenyl, C₂₋₆ alkynyl, C₃₋₁₀ cycloalkyl (e.g. adamantyl), and benzyl, wherein each R¹¹ and R¹², when not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl (e.g. methyl or ethyl), C₃₋₁₀ cycloalkyl (e.g. adamantyl), and benzyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

For example, R¹¹ and R¹² are independently selected from H and C₁₋₆ alkyl (e.g. methyl or ethyl), C₃₋₁₀ cycloalkyl (e.g. adamantyl), and benzyl; wherein each R¹¹ and R¹², when not H, are independently optionally substituted with 1 or 2 —R^{β}.

For example, R¹¹ is H, and R¹² is selected from H and C₁₋₆ alkyl (methyl or ethyl), C₃₋₁₀ cycloalkyl (e.g. adamantyl), and benzyl; wherein R¹², when not H, is optionally substituted with 1 —R^{β}.

For example, R¹¹ and R¹² are both H.

For example, R¹¹ is H, and R¹² is benzyl, optionally substituted with 1 —R^{β}. For example, R¹¹ is H, and R¹² is benzyl substituted with methoxy, e.g. R¹² is ortho-methoxy-benzyl.

When R¹¹ and R¹² together form a 5- or 6-membered heterocycle as described above, it may be a 5- or 6-membered heterocycle optionally having one additional heteroatom selected from N and O; wherein the 5- or 6-membered heterocycle is optionally substituted with 1 or 2 C₁₋₄ alkyl or benzyl. In this respect, the 5- or 6-membered heterocycle may be morpholine, piperidine, piperazine, or pyrrolidine optionally substituted with 1 or 2 C₁₋₄ alkyl or benzyl. For example, the 5- or 6-membered heterocycle may be morpholine, piperazine, 4-methyl piperazine, or pyrrolidine. In one embodiment, n is an integer from 1 to 4.

For example, n may be 3 or 4.

For example, n may be 1.

Each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₂ alkyl and —O(C₁₋₂ alkyl), and any —R^{β} may optionally be substituted with one or more halo, —OH, —NH₂, —CN, or —NO₂ groups.

Each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH,, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

For example, each R¹³ is independently selected from a H and C₁-C₃ alkyl, wherein any —R¹³ may optionally be substituted with one or more —R¹⁴.

For example, each R¹³ is independently selected from a H and C₁-C₂ alkyl.

Each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

For example, each R¹⁴ is independently selected from a C₁-C₆ alkyl, halo, —NO₂, —CN, —OH, —NH,, mercapto, formyl, carboxy, carbamoyl, and C₁₋₆ alkoxy, wherein any —R₁₄ may optionally be substituted with one or more —R₁₅.

Each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

For example, each —R¹⁵ is independently selected from halogen, nitro, cyano, hydroxy, trifluoromethoxy, trifluoromethyl, amino, formyl, and carboxy.

Each p is independently an integer selected from 1 to 4.

For example, each p is independently an integer selected from 2 to 4.

For example, each p is independently selected from 3 and 4.

Each q is independently an integer selected from 1 to 4.

For example, each q is independently an integer selected from 2 to 4.

For example, each q is independently selected from 3 and 4.

For example, Z may be —N(R¹⁰)—(CH₂)₃—N(R¹⁰)—(CH₂)₄—NR¹¹R¹².

For example, Z may be —N(R¹⁰)—(CH₂)₄—N(R¹⁰)—(CH₂)₃—NR¹¹R¹².

In one embodiment, n is 1, for example as represented by Formula (2) below.

For example, the invention provides a compound of formula (2):

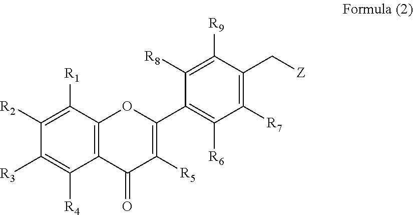

wherein R¹-R⁹ and Z are as defined herein.

In one embodiment, the compound may be a compound of formula (2):

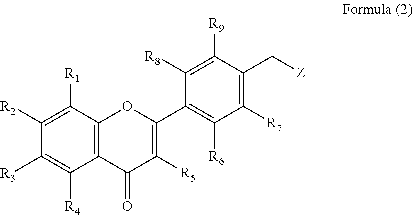

wherein:
Z is selected from: —NR¹¹R¹²;
   —N(R¹⁰)—(CH₂)ₚ—NR¹¹R¹²; and
   —N(R¹⁰)—(CH₂)_{q}—N(R¹⁰)—CH₂)_{q}—NR¹¹R¹²;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl , —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl, or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-6;
each p is independently an integer selected from 1 to 4; and
each q is independently an integer selected from 1 to 4.

R¹ and R², independently, are selected from —OH, —O—C₁₋₄ alkyl, —OC(O)R₁₃, —OC(O)NHR¹³, —OC(O)N(R¹³)₂. In one embodiment, R¹ and R², independently, are selected from —OH, and —O—C₁₋₄ alkyl. In one embodiment, R¹ and R², independently, are selected from —OH and —OCH₃.

In one embodiment, R¹ and R² together form —O—CH₂—O—.

R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. In one embodiment, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}. In one embodiment, R³, R⁴, R⁵, R⁶, R⁷, R⁸, and R⁹, independently, are selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}. In one embodiment, R³, R⁴, R⁵, R⁶, R⁷, R⁸, and R⁹, independently, are selected from H; halo; —CN; —NO₂; and —NH₂. In one embodiment, R³, R⁴, R⁵, R⁶, R⁷, R⁸, and R⁹, are H.

In one embodiment, R¹ and R², independently, are selected from —OH, —O—C₁₋₄ alkyl, —OC(O)R₁₃, —OC(O)NHR¹³, —OC(O)N(R¹³)₂; and R³-R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹ and R², independently, are selected from —OH, and —O—C₁₋₄ alkyl. For example, R³-R⁹ are independently selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —O—C₁₋₄ alkyl. For example, R³-R⁹ are independently selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and −OCH₃. For example, R³-R⁹, are independently selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹ and R², independently, are selected from —OH, and —OCH₃. For example, R³-R⁹, are independently selected from H; halo; —CN; —NO₂; and —NH₂.

In one embodiment, R¹ and R², independently, are selected from —OH, and —OCH₃. For example, R³-R⁹, are H.

In one embodiment, R¹ and R² are independently selected from —OH, —O—C₁₋₄ alkyl, —OC(O)R₁₃, —OC(O)NHR¹³, —OC(O)N(R¹³)₂; R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R² are independently selected from —OH and —O—C₁₋₄ alkyl, e.g. —OH and —OCH₃; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R² are independently selected from —OH and —O—C₁₋₄ alkyl; and R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ , and R², independently, are selected from —OH and —OCH₃; and R³, R⁴, R⁵, R⁶, R⁷, R⁸, and R⁹, independently, are selected from H; halo; —CN; —NO₂; —SH; —SO₂H; and —NH₂. For example, R³, R⁴, R⁵, R⁶, R⁷, R⁸, and R⁹ are H.

In one embodiment, R¹ is —O—C₁₋₄ alkyl, e.g. —O—Me; R² is OH; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, R¹ and R² are OH; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁵, R⁶, R⁷, R⁸, and R⁹, independently, are selected from H; halo; —CN; —NO₂; —SH; —SO₂H; —NH₂; —CHO; —COOH. For example, R³, R⁴, R⁵, R⁶, R⁷, R⁸, and R⁹ are H.

In one embodiment, Z is —NR¹¹R¹².

In one embodiment, Z is —N(R¹⁰)—(CH₂)ₚ—NR¹¹R¹².

In one embodiment, Z is —N(R¹⁰)—(CH₂)_{q}—N(R¹⁰)—(CH₂)_{q}—NR¹¹R¹².

Each R¹⁰ is independently selected from H, C₁₋₆ alkyl, C₂-C₆ alkenyl, C₂₋₆ alkynYl, C₃₋₁₀ cycloalkyl, and benzyl, wherein each Rio, when not H, is independently optionally substituted with 1 or 2 —R^{β}.

For example, each R¹⁰ may independently be selected from H, C₁₋₆ alkyl, and C₂-C₄ alkenyl.

For example, each R¹⁰ may independently be selected from H, C₁₋₃ alkyl, and C₂-C₄ alkenyl.

For example, each R¹⁰ is independently selected from H and C₁₋₆ alkyl.

For example, each R¹⁰ may independently be selected from H and C₁₋₃ alkyl.

For example, each R¹⁰ may independently be selected from H and —CH₃. R¹¹ and R¹² are independently selected from H, C₁₋₆-alkyl (e.g. methyl or ethyl), C₂-C₆ alkenyl, C₂₋₆ alkynyl, C₃₋₁₀ cycloalkyl (e.g. adamantyl), and benzyl, wherein each R¹¹ and R¹², when not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl (e.g. methyl or ethyl), C₃₋₁₀ cycloalkyl (e.g. adamantyl), and benzyl ; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

For example, R¹¹ and R¹² are independently selected from H and C₁₋₆ alkyl (e.g. methyl or ethyl), C₃₋₁₀ cycloalkyl (e.g. adamantyl), and benzyl; wherein each R¹¹ and R¹², when not H, are independently optionally substituted with 1 or 2 —R^{β}.

For example, R¹¹ is H, and R¹² is selected from H and C₁₋₆ alkyl (methyl or ethyl), C₃₋₁₀ cycloalkyl (e.g. adamantyl), and benzyl; wherein R¹², when not H, is optionally substituted with 1 —R^{β}.

For example, R¹¹ and R¹² are both H.

For example, R¹¹ is H, and R¹² is benzyl, optionally substituted with 1 —R^{β}. For example, R¹¹ is H, and R¹² is benzyl substituted with methoxy, e.g. R¹² is ortho-methoxy-benzyl.

When R¹¹ and R¹² together form a 5- or 6-membered heterocycle as described above, it may be a 5- or 6-membered heterocycle optionally having one additional heteroatom selected from N and O; wherein the 5- or 6-membered heterocycle is optionally substituted with 1 or 2 C₁₋₄ alkyl or benzyl. In this respect, the 5- or 6-membered heterocycle may be morpholine, piperidine, piperazine, or pyrrolidine optionally substituted with 1 or 2 C₁₋₄ alkyl or benzyl. For example, the 5- or 6-membered heterocycle may be morpholine, piperazine, 4-methyl piperazine, or pyrrolidine.

In one embodiment, n is an integer from 1 to 4.

For example, n may be 3 or 4.

For example, n may be 1.

Each —R^{β} is independently selected from a C₁-C₆ alkyl, C₂-C₆ alkenyl, C₂-C₆ alkynyl , —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁₋₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₂ alkyl and —O(C₁₋₂ alkyl), and any —R^{β} may optionally be substituted with one or more halo, —OH, —NH₂, —CN, or —NO₂ groups.

Each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

For example, each R¹³ is independently selected from a H and C₁-C₃ alkyl, wherein any —R¹³ may optionally be substituted with one or more —R¹⁴.

For example, each R¹³ is independently selected from a H and C₁-C₂ alkyl.

Each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

For example, each R¹⁴ is independently selected from a C₁-C₆ alkyl, halo, —NO₂, —CN, —OH, —NH₂, mercapto, formyl, carboxy, carbamoyl, and C₁₋₆ alkoxy, wherein any —R₁₄ may optionally be substituted with one or more —R₁₅.

Each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

For example, each —R¹⁵ is independently selected from halogen, nitro, cyano, hydroxy, trifluoromethoxy, trifluoromethyl, amino, formyl, and carboxy.

Each p is independently an integer selected from 1 to 4.

For example, each p is independently an integer selected from 2 to 4.

For example, each p is independently selected from 3 and 4.

Each q is independently an integer selected from 1 to 4.

For example, each q is independently an integer selected from 2 to 4.

For example, each q is independently selected from 3 and 4.

For example, Z may be —N(R¹⁰)—(CH₂)₃—N(R¹⁰)—(CH₂)₄—NR¹¹R¹².

For example, Z may be —N(R¹⁰)—(CH₂)₄—N(R¹⁰)—(CH₂)₃—NR¹¹R¹².

A second aspect of the invention provides a compound selected from the group consisting of:

TABLE A

| | |
|---|---|
| 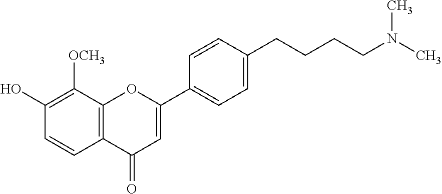 [SMILES: COc1c(O)ccc2c(=O)cc(-c3ccc(CCCCN(C)C)cc3)oc12] | SND 141 |
| 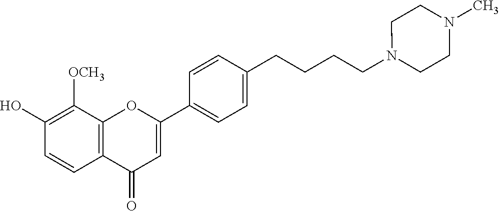 [SMILES: COc1c(O)ccc2c(=O)cc(-c3ccc(CCCCN4CCN(C)CC4)cc3)oc12] | SND 142 |
| 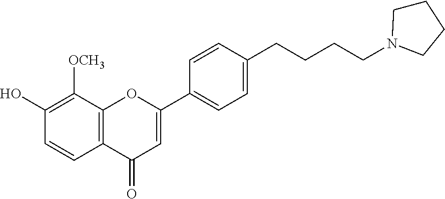 [SMILES: COc1c(O)ccc2c(=O)cc(-c3ccc(CCCCN4CCCC4)cc3)oc12] | SND 143 |
| 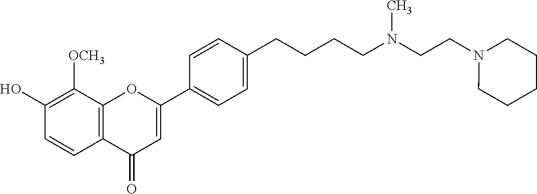 [SMILES: COc1c(O)ccc2c(=O)cc(-c3ccc(CCCCN(C)CCN4CCCCC4)cc3)oc12] | SND 146 |
| 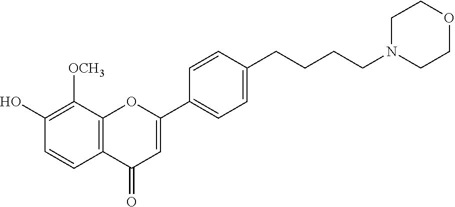 [SMILES: COc1c(O)ccc2c(=O)cc(-c3ccc(CCCCN4CCOCC4)cc3)oc12] | SND 147 |
| 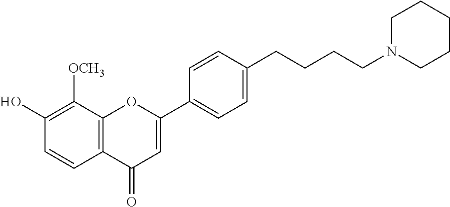 [SMILES: COc1c(O)ccc2c(=O)cc(-c3ccc(CCCCN4CCCCC4)cc3)oc12] | SND 148 |
| 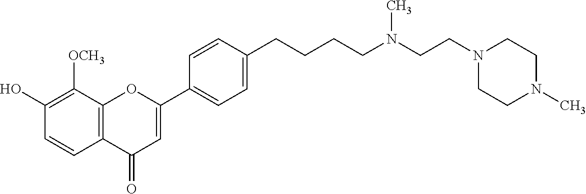 [SMILES: COc1c(O)ccc2c(=O)cc(-c3ccc(CCCCN(C)CCN4CCN(C)CC4)cc3)oc12] | SND 149 |
| 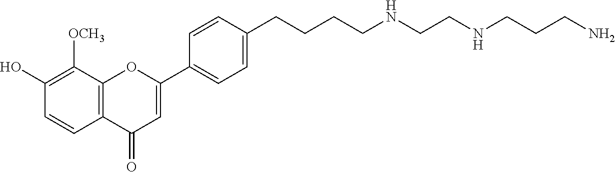 [SMILES: COc1c(O)ccc2c(=O)cc(-c3ccc(CCCCNCCNCCCN)cc3)oc12] | SND 211 |
| 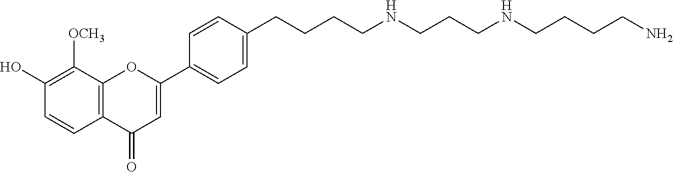 [SMILES: COc1c(O)ccc2c(=O)cc(-c3ccc(CCCCNCCCNCCCCN)cc3)oc12] | SND 212 |
| 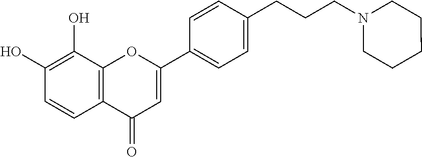 [SMILES: O=c1cc(-c2ccc(CCCN3CCCCC3)cc2)oc2c(O)c(O)ccc12] | SND 172 |
| 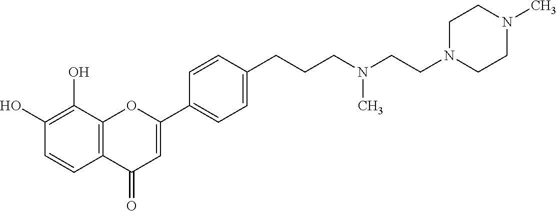 [SMILES: CN(CCCc1ccc(-c2cc(=O)c3ccc(O)c(O)c3o2)cc1)CCN1CCN(C)CC1] | SND 173 |
| 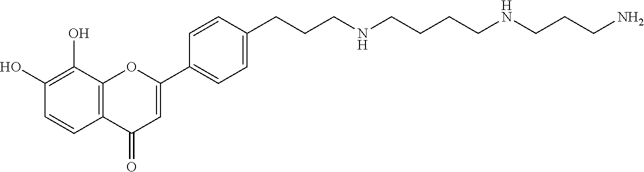 [SMILES: NCCCNCCCCNCCCc1ccc(-c2cc(=O)c3ccc(O)c(O)c3o2)cc1] | SND 174 |
| 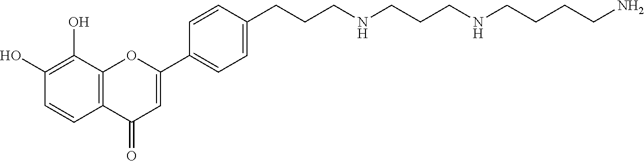 [SMILES: NCCCCNCCCNCCCc1ccc(-c2cc(=O)c3ccc(O)c(O)c3o2)cc1] | SND 175 |
| 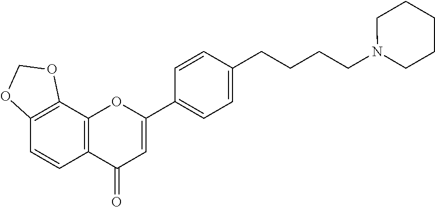 [SMILES: O=c1cc(-c2ccc(CCCCN3CCCCC3)cc2)oc2c3c(ccc12)OCO3] | SND 390 |
| 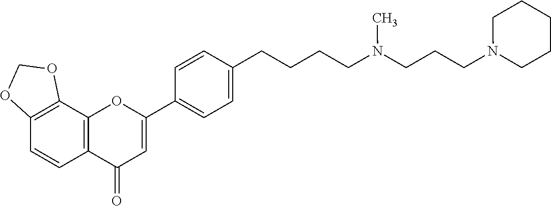 [SMILES: CN(CCCCc1ccc(-c2cc(=O)c3ccc4c(c3o2)OCO4)cc1)CCCN1CCCCC1] | SND 391 |
| 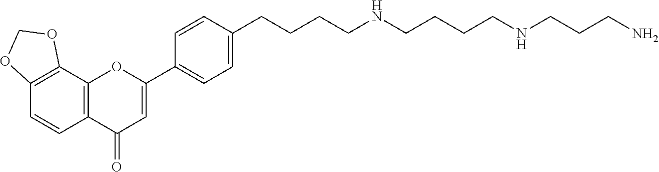 [SMILES: NCCCNCCCCNCCCCc1ccc(-c2cc(=O)c3ccc4c(c3o2)OCO4)cc1] | SND 392 |
| 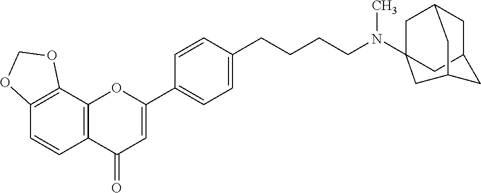 [SMILES: CN(CCCCc1ccc(-c2cc(=O)c3ccc4c(c3o2)OCO4)cc1)C12CC3CC(CC(C3)C1)C2] | SND 393 |
| 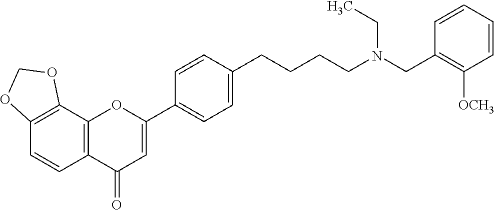 [SMILES: CCN(CCCCc1ccc(-c2cc(=O)c3ccc4c(c3o2)OCO4)cc1)Cc1ccccc1OC] | SND 394 |
| 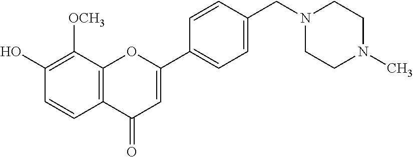 [SMILES: COc1c(O)ccc2c(=O)cc(-c3ccc(CN4CCN(C)CC4)cc3)oc12] | SND 160 |
| 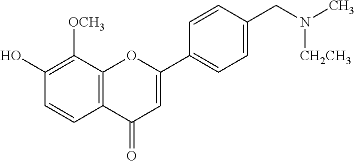 [SMILES: CCN(C)Cc1ccc(-c2cc(=O)c3ccc(O)c(OC)c3o2)cc1] | SND 161 |
| 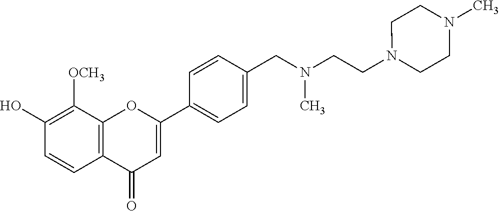 [SMILES: COc1c(O)ccc2c(=O)cc(-c3ccc(CN(C)CCN4CCN(C)CC4)cc3)oc12] | SND 162 |
| 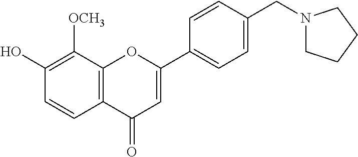 [SMILES: COc1c(O)ccc2c(=O)cc(-c3ccc(CN4CCCC4)cc3)oc12] | SND 163 |
| 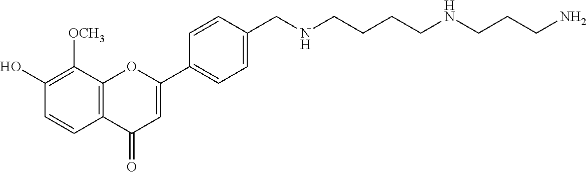 [SMILES: COc1c(O)ccc2c(=O)cc(-c3ccc(CNCCCCNCCCN)cc3)oc12] | SND 164 |
| 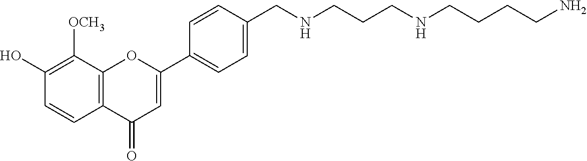 [SMILES: COc1c(O)ccc2c(=O)cc(-c3ccc(CNCCCNCCCCN)cc3)oc12] | SND 165 |
| 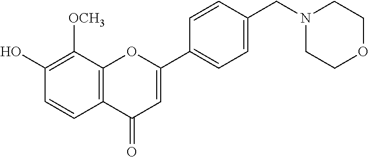 [SMILES: COc1c(O)ccc2c(=O)cc(-c3ccc(CN4CCOCC4)cc3)oc12] | SND 166 |
| 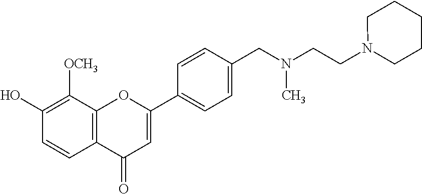 [SMILES: COc1c(O)ccc2c(=O)cc(-c3ccc(CN(C)CCN4CCCCC4)cc3)oc12] | SND 167 |
| 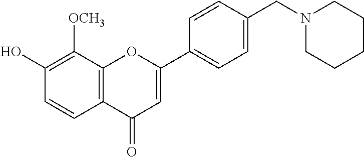 [SMILES: COc1c(O)ccc2c(=O)cc(-c3ccc(CN4CCCCC4)cc3)oc12] | SND 168 |

A third aspect of the invention provides pharmaceutically acceptable salt, multi-salt, solvate or prodrug of the compound of the first or second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C , ¹³C , ¹H, ²H (D), ¹⁴N, ¹⁵N , ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition.

Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In one embodiment of the fifth, sixth, or seventh aspect of the present invention, the disease, disorder or condition is selected from but not limited to: breast cancer, brain cancer, lung cancer, leukaemia, lymphoma, melanoma, ovarian cancer, renal cancer, prostate cancer and pancreatic cancer. The cancers may include resistant types of such tumors.

In general embodiments, the disease, disorder or condition is cancer.

In one embodiment the cancer is brain cancer.

In one embodiment the cancer is breast cancer.

In one embodiment the cancer is colon cancer.

In one embodiment the cancer is leukaemia.

In one embodiment the cancer is lymphoma.

In one embodiment the cancer is lung cancer.

In one embodiment the cancer is melanoma.

In one embodiment the cancer is ovarian cancer.

In one embodiment the cancer is pancreatic cancer.

In one embodiment the cancer is prostate cancer.

In one embodiment the cancer is renal cancer.

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

### General Synthesis of Compounds SND141-148

Structure of the SND derivatives and the codes used in the chemical synthesis

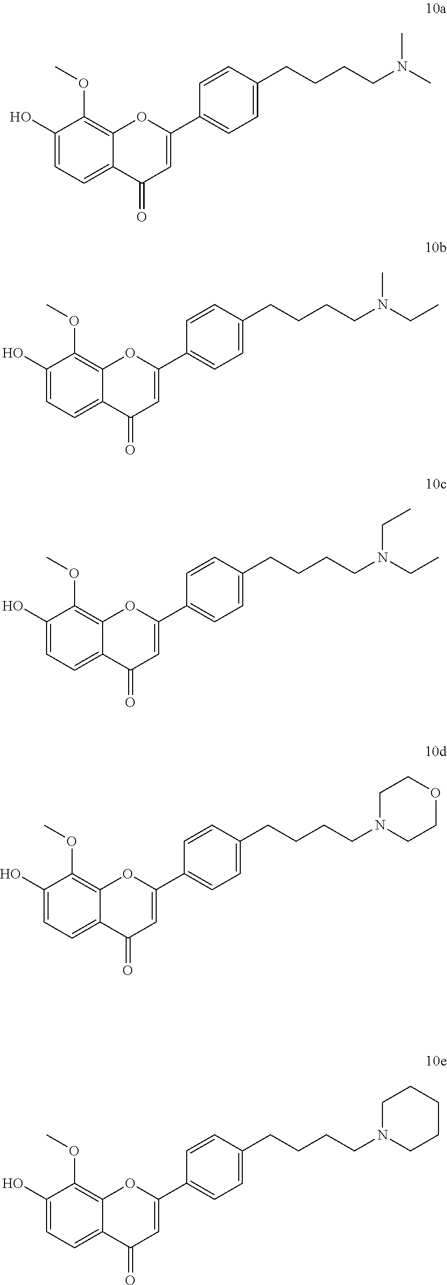 [SMILES: COc1c(O)ccc2c(=O)cc(-c3ccc(CCCCN(C)C)cc3)oc12 ; CCN(C)CCCCc1ccc(-c2cc(=O)c3ccc(O)c(OC)c3o2)cc1 ; CCN(CC)CCCCc1ccc(-c2cc(=O)c3ccc(O)c(OC)c3o2)cc1 ; COc1c(O)ccc2c(=O)cc(-c3ccc(CCCCN4CCOCC4)cc3)oc12 ; COc1c(O)ccc2c(=O)cc(-c3ccc(CCCCN4CCCCC4)cc3)oc12]

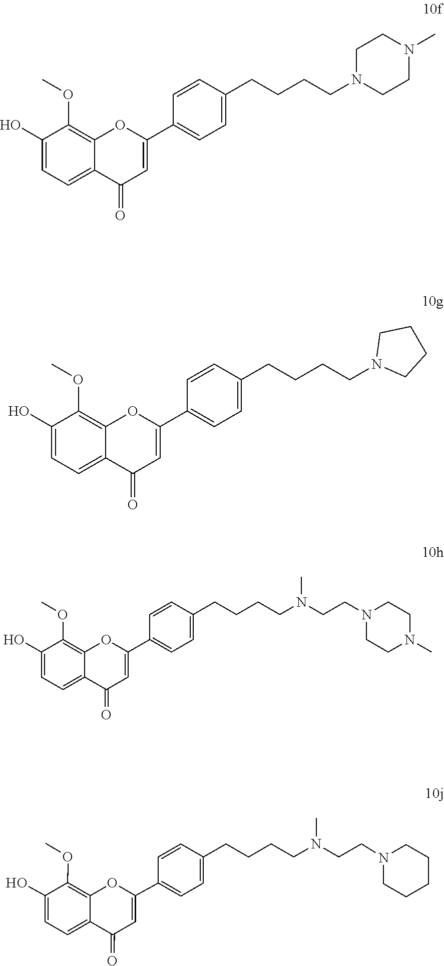 [SMILES: COc1c(O)ccc2c(=O)cc(-c3ccc(CCCCN4CCN(C)CC4)cc3)oc12 ; COc1c(O)ccc2c(=O)cc(-c3ccc(CCCCN4CCCC4)cc3)oc12 ; COc1c(O)ccc2c(=O)cc(-c3ccc(CCCCN(C)CCN4CCN(C)CC4)cc3)oc12 ; COc1c(O)ccc2c(=O)cc(-c3ccc(CCCCN(C)CCN4CCCCC4)cc3)oc12]

Scheme 1 represents the chemical synthesis of amines 10a-j.

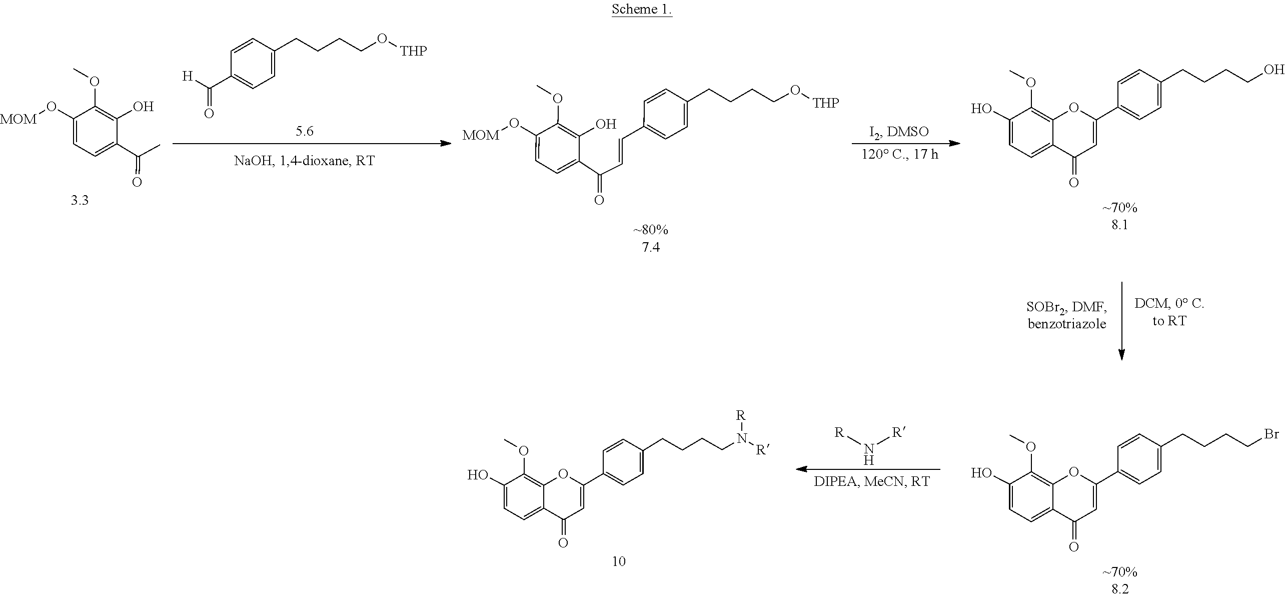

Flavones 10a-j were prepared following the synthesis route depicted in Scheme 1. The flavone scaffold was synthesized by Claisen-Schmidt condensation between substituted acetophenone 3.3 and benzaldehyde derivative 5.6, followed by cyclization with iodine to give 8.1. Alcohol 8.1 was brominated with SOBr₂/DMF/benzotriazole mixture, by using a modified procedure from a published patent Patent WO201601446 to give 8.2 in 55-70% yield. Benzotriazole was added to the reaction mixture to avoid bromination of additional positions.

A robust method using Hünig's base and acetonitrile was chosen for the synthesis of tertiary amines from corresponding secondary amines and alkyl halides [Arkivoc, 2005, 6, 287-292].

This method has previously proven to yield pure product in good to excellent yields. Originally this method used 1.1 eq of alkyl halide, but since the bromide 8.2 was the limiting substrate, considerably more equivalents of the secondary amines were used. At least 5 eq of the secondary amine was used, and in the case of diethylamine in total 30 eq was used and even that was not enough to drive the reaction to completion. At first, the reaction mixture was too dilute, prolonging the reaction time to more than 24 h. In order to minimize chances of quarternalization of the amine, the halide 8.2 was added to the solution of DiPEA and the corresponding secondary amine, in that case there was always an excess of amine relative to the halide. Since the starting material was not soluble in acetonitrile, a slurry of 8.2 was added to the amines, which quickly dissolved in the mixture of amines and in some cases the product started to precipitate out.

### (E)-1-(2-Hydroxy-3-methoxy-4-(methoxymethoxy)phenyl)-3-(4-(4-((tetrahydro-2H-pyran-2-yl)oxy)butypphenyl)prop-2-en-1-one (7.4)

Sodium hydroxide (35 g, 55 Eq, 0.88 mol) in water (35 mL) was added at room temperature to a solution of 1-(2-hydroxy-3-methoxy-4-(methoxymethoxy)phenyl)ethan-1-one (3.616 g, 1 Eq, 15.98 mmol) and 4-(4-((tetrahydro-2H-pyran-2-yl)oxy)butyl)benzaldehyde (5.048 g, 1.204 Eq, 19.24 mmol) in 1,4-dioxane (75 mL) was added. The reaction mixture was stirred vigorously for 22 hours at room temperature. The dark reaction mixture was diluted with 50 mL of water, cooled to 0° C. and neutralized with approximately 55 g of citric acid until pH was neutral. The orange mixture was further diluted with 200 mL of water and extracted with 3×100 mL of DCM. The last fraction had already very little UV-activity. The organic layers were combined and washed with 200 mL of brine, which in turn was extracted with 2×50 mL of DCM. Organic fractions were combined, dried with sodium sulfate, filtered through paper filter and evaporated to dryness. Resultant 8.952 g of orange oil was purified by normal phase flash-chromatography using ethyl acetate/heptane as eluent. 6.489 g of (E)-1-(2-hydroxy-3-methoxy-4-(methoxymethoxy)phenyl)-3-(4-(4-((tetrahydro-2H-pyran-2-yeoxy)butyl)phenyeprop-2-en-1-one (6.489 g, 13 mmol, 82% yield, 95% purity) as an orange oil was obtained.

### 7-Hydroxy-2-(4-(4-hydroxybutyl)phenyl)-8-methoxy-4H-chromen-4-one (8.1)

A stirred solution of (E)-1-(2-hydroxy-3-methoxy-4-(methoxymethoxy)phenyl)-3-(4-(4-((tetrahydro-2H-pyran-2-yeoxy)butyl)phenyeprop-2-en-1-one (5.6 g, 1 Eq, 12 mmol) and iodine (0.439 g, 0.15 Eq, 1.73 mmol) in DMSO (100 mL) was heated to 120° C. for 17 hours. The dark mixture was allowed to cool to room temperature and poured into 700 mL of water. The resultant beige suspension was extracted with 6×200 mL of EtOAc. The combined organic fractions were washed with 400 mL of 10% Na₂S₂O₃, which in turn was extracted with 50 mL of EtOAc. Organic layers were combined and washed with 400 mL of water, the aqueous layer was further extracted with 50 mL of EtOAc. The combined organic fractions were washed with 400 mL of brine, dried with Na₂SO₄, filtered through paper filter and evaporated to dryness until the crude product solidified. 7-Hydroxy-2-(4-(4-hydroxybutyl)phenyl)-8-methoxy-4H-chromen-4-one (4.1481 g, 9.7 mmol, 82% yield, 80% purity) was obtained as a brown solid and used in the next step without further purification.

### 2-(4-(4-Bromobutyl)phenyl)-7-hydroxy-8-methoxy-4H-chromen-4-one (8.2)

7-Hydroxy-2-(4-(4-hydroxybutyl)phenyl)-8-methoxy-4H-chromen-4-one (4.148 g, 1.0 Eq, 12.19 mmol) and DCM (150 mL) were transferred to a dried 250 mL three-neck round-bottom flask under nitrogen flow. The resultant suspension was cooled to 0° C., before 1H-benzo[d][1,2,3]triazole (1.894 g, 1.305 Eq, 15.90 mmol) and N,N-dimethylformamide (0.20 g, 0.21 mL, 0.22 Eq, 2.7 mmol) was added under nitrogen flow. After that, sulfurous dibromide (2.9 g, 1.1 mL, 1.2 Eq, 14 mmol) was added drop-wise in 2 min to the cooled suspension. The mixture was stirred for another 3 min at 0° C. before allowing it to warm to room temperature. The mixture was stirred at 20° C. for 16 hours, before a sample from the reaction mixture was treated with EtOAc and sat. NaHCO₃ and analyzed by LC-MS. If very little or no starting material could be seen, the reaction mixture was basified with 150 mL of sat. NaHCO₃ and the aqueous layer was extracted with 8×100 mL of DCM. The organic layers were combined and washed with 300 mL of brine. The brine layer was in turn extracted with 2×50 mL of DCM. The organic fractions were combined and dried with Na₂SO₄, filtered and evaporated to dryness. The resultant 6.725 g of crude material was purified by normal phase flash-chromatography using DCM:MeOH eluent system to give 2-(4-(4-bromobutyl)phenyl)-7-hydroxy-8-methoxy-4H-chromen-4-one (2.891 g, 6.7 mmol, 55% yield, 93% purity) as a grey-brown powder.

### General Procedure for Alkylation of Secondary Amines

A slurry of 2-(4-(4-bromobutyl)phenyl)-7-hydroxy-8-methoxy-4H-chromen-4-one (8.2) (1 Eq) in MeCN was added under nitrogen flow to a stirring solution of DiPEA (1.5 Eq) and secondary amine (5-10 Eq) in MeCN in a dried round-bottom flask. The mixture was allowed to stir at room temperature for 16 h or more until no starting material was left, based on TLC or LC-MS. The mixture was then evaporated to dryness, dissolved in DCM and washed with brine:water 1:1 mixture. The aqueous layer was extracted with DCM until no UV-activity could be seen on a TLC plate. The organic layers were combined, dried with Na₂SO₄, filtered and evaporated to dryness. The crude product was purified byflash-chromatography and dried in vacuum. Alternatively, for the less soluble series 11 compounds, filtration of the reaction mixture was used instead of an aqueous work-up and this gave mostly pure products.

### 2-(4-(4-(Dimethylamino)butyl)phenyl)-7-hydroxy-8-methoxy-4H-ehromen-4-one (10a/SND141)

2-(4-(4-Bromobutyl)phenyl)-7-hydroxy-8-methoxy-4H-chromen-4-one (8.2) (507 mg, 1 Eq, 1.26 mmol) in MeCN (5 mL) was reacted with dimethylamine (0.57 g, 6.3 mL, 2 molar, 10 Eq, 13 mmol) in THF (6 mL) according to the general procedure. Purification by normal phase flash-chromatography using DCM:NH₃ in MeOH eluent system. After chromatography, excess water was removed azeotropically with 10 mL of toluene and drying in vacuum, 2-(4-(4-(dimethylamino)butyl)phenyl)-7-hydroxy-8-methoxy-4H-chromen-4-one (304 mg, 0.79 mmol, 63% yield, 95% purity) was obtained as a fine yellow powder.

### 7-Hydroxy-8-methoxy-2-(4-(4-morpholinobutypphenyl)-4H-chromen-4-one (10d/SND147)

2-(4-(4-Bromobutyl)phenyl)-7-hydroxy-8-methoxy-4H-chromen-4-one (8.2) (634 mg, 1 Eq, 1.57 mmol) was reacted morpholine (657 mg, 650 μL, 4.79 Eq, 7.54 mmol) according to the general procedure. Purification by normal phaseflash-chromatography, using DCM:NH₃ in MeOH eluent system, yielding after removal of water azeotropically with toluene and drying in vacuum, 7-hydroxy-8-methoxy-2-(4-(4-morpholinobutyl)phenyl)-4H-chromen-4-one (478.6 mg, 1.1 mmol, 72% yield, 97% Purity) as a light beige powder.

### 7-Hydroxy-8-methoxy-2-(4-(4-(piperidin-1-yl)butypphenyl)-4H-chromen-4-one (10e/SND148)

2-(4-(4-Bromobutyl)phenyl)-7-hydroxy-8-methoxy-4H-chromen-4-one (489.4 mg, 1 Eq, 1.214 mmol) was reacted with piperidine (517 mg, 600 μL, 5.01 Eq, 6.07 mmol) according to the general procedure. Purification by normal phaseflash-chromatography, using DCM:NH₃ in MeOH eluent system, yielding after removal of traces of water azeotropically by toluene and drying in vacuum 7-hydroxy-8-methoxy-2-(4-(4-(piperidin-1-yl)butyl)phenyl)-4H-chromen-4-one (0.378 g, 0.90 mmol, 74% yield, 97% purity) as a yellow solid.

### 7-Hydroxy-8-methoxy-2-(4-(4-(4-methylpiperazin-1-yl)butyl)phenyl)-4H-chromen-4-one (10f/SND142)

2-(4-(4-Bromobutyl)phenyl)-7-hydroxy-8-methoxy-4H-chromen-4-one (8.2) (685 mg, 1 Eq, 1.70 mmol) was reacted with 1-methylpiperazine (361 mg, 400 μL, 2.12 Eq, 3.61 mmol) according to the general procedure. Purification by normal phaseflash-chromatography, using DCM:NH₃ in MeOH eluent system, yielding after drying in vacuum 7-hydroxy-8-methoxy-2-(4-(4-(4-methylpiperazin-1-yebutyl)phenyl)-4H-chromen-4-one (502 mg, 1.1 mmol, 66% yield, 95% purity) as a beige powder.

### 7-Hydroxy-8-methoxy-2-(4-(4-(Pyrrolidin-1-yl)butyl)phenyl)-4H-chromen-4-one (10g/SND143)

2-(4-(4-Bromobutyl)phenyl)-7-hydroxy-8-methoxy-4H-chromen-4-one (405.5 mg, 1 Eq, 1.006 mmol) was reacted with pyrrolidine (368 mg, 425 μL, 5.15 Eq, 5.18 mmol) according to the general procedure. Purification by normal phaseflash-chromatography, using DCM:NH₃ in MeOH eluent system, yielding after removal of traces of water azeotropically by toluene and drying in vacuum 7-hydroxy-8-methoxy-2-(4-(4-(pyrrolidin-1-yl)butyl)phenyl)-4H-chromen-4-one (250 mg, 0.61 mmol, 61% yield, 96% purity) as a yellow powder.

### 7-Hydroxy-8-methoxy-2-(4-(4-(methyl(2-(piperidin-1-yl)ethyl)amino)butyl)phenyl)-4H-chromen-4-one (10h/SND146)

2-(4-(4-Bromobutyl)phenyl)-7-hydroxy-8-methoxy-4H-chromen-4-one (122 mg, 1 Eq, 303 μmol) was reacted with N-methyl-2-(piperidin-1-yl)ethan-1-amine (360 mg, 8.37 Eq, 2.53 mmol) according to the general procedure. Purification by normal phaseflash-chromatography, using silica and aluminum oxide as stationary phases. After drying, 7-hydroxy-8-methoxy-2-(4-(4-(methyl(2-(piperidin-1-yl)ethyl)amino)butyl)phenyl)-4H-chromen-4-one (65 mg, 0.13 mmol, 44% yield, 96% purity) was obtained as an orange amorphous solid.

### 7-Hydroxy-8-methoxy-2-(4-(4-(methyl(2-(4-methylpiperazin-1-yl)ethyl)amino)butyl)phenyl)-4H-chromen-4-one trihydrochloride (10j/SND149)

2-(4-(4-bromobutyl)phenyl)-7-hydroxy-8-methoxy-4H-chromen-4-one (0.343 g, 1 Eq, 851 μmol) was reacted with N-methyl-2-(4-methylpiperazin-1-yl)ethan-1-amine (601 mg, 4.49 Eq, 3.82 mmol) according to the general procedure. Purification by normal phaseflash-chromatography, using aluminum oxide as stationary phase, and once by preparative HPLC to give 7-hydroxy-8-methoxy-2-(4-(4-(methyl(2-(4-methylpiperazin-1-yl)ethyl)amino)butyl)phenyl)-4H-chromen-4-one (74 mg, 0.15 mmol, 18% yield, 97% Purity). The free base was converted to the trihydrochloric acid salt by dissolving it in minimum amount of DCM and treating it with several equivalents of 4 M HCl in 1,4-dioxane. After 1 h, the mixture was diluted with Et₂O, the solution was removed and the yellow precipitate was washed with 3×1 mL of Et₂O and dried. The HCl salt was obtained as a yellow powder.

### Synthesis of SND170-175 & 177

The following scheme was employed to synthesise SND170,

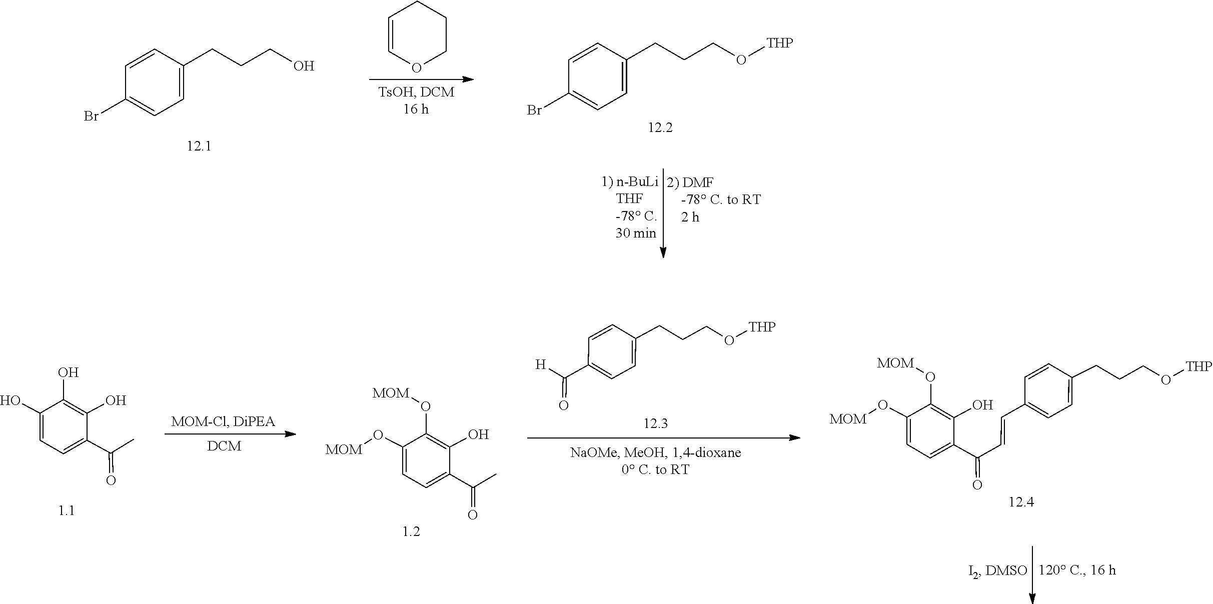

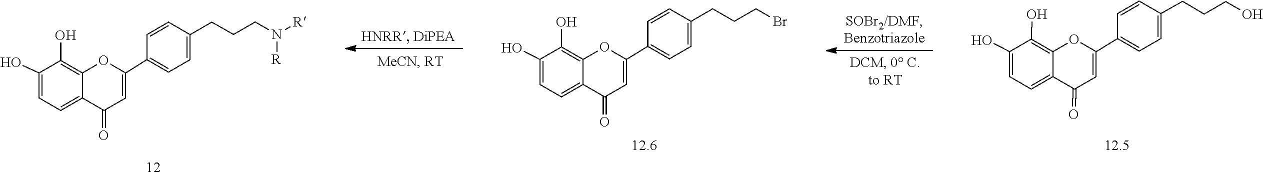

The following general scheme was employed to synthesise SND171-175 and 177.

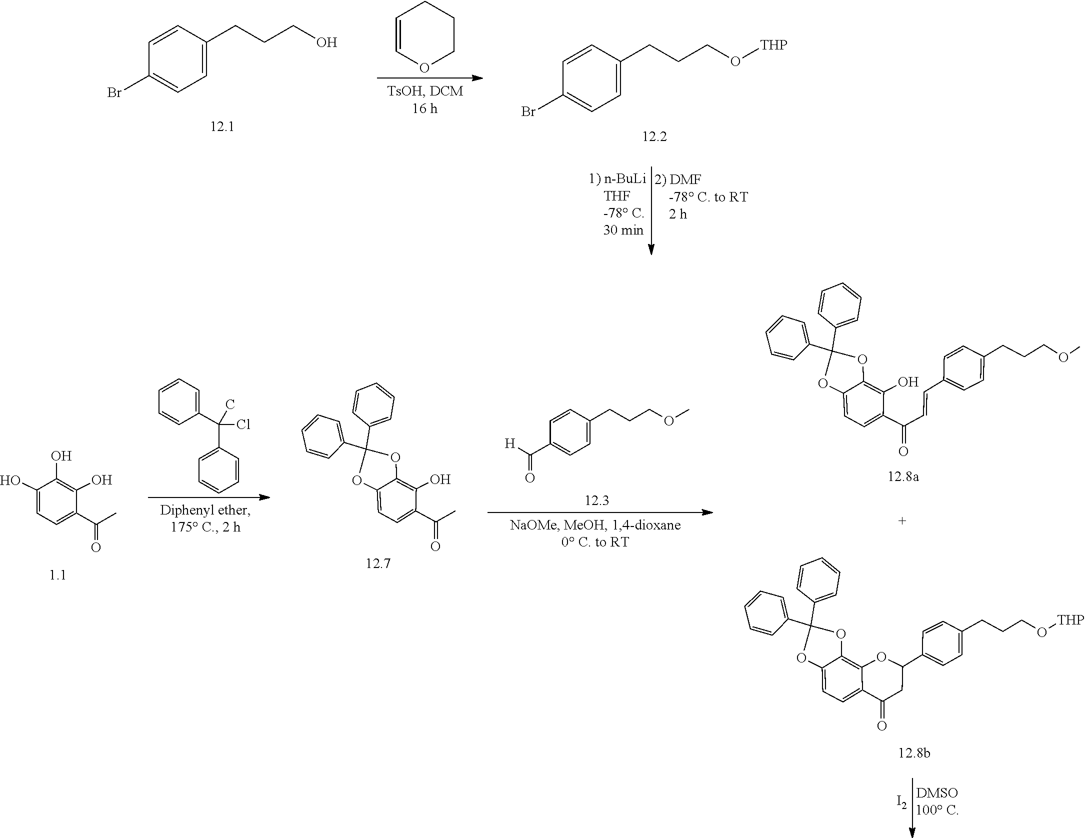

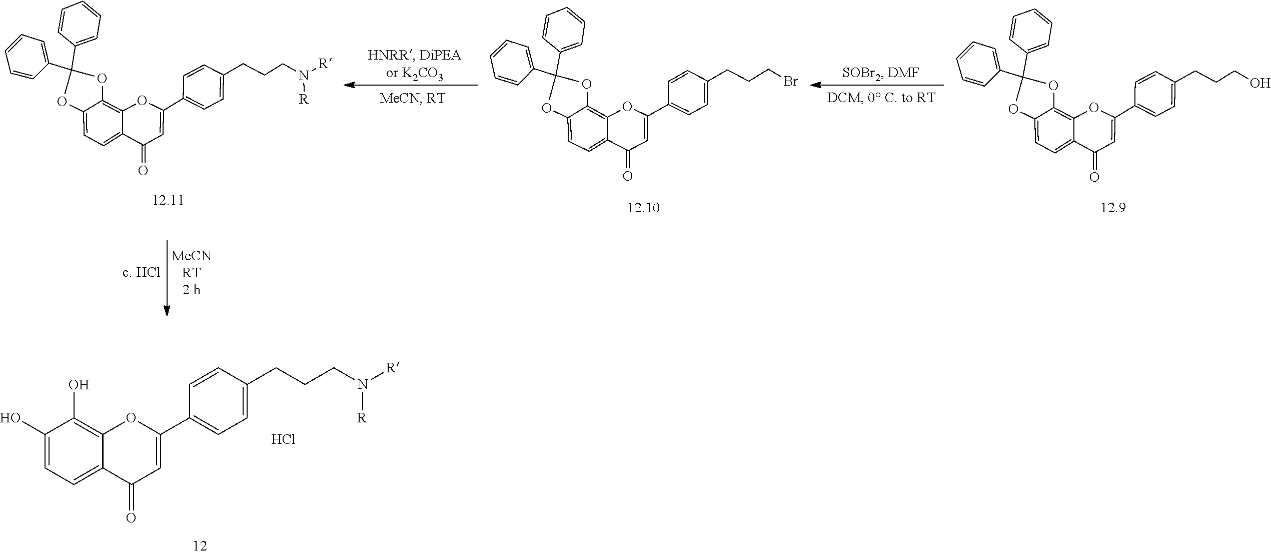

### 1-(2-Hydroxy-3,4-bis(methoxymethoxy)phenyl)ethan-1-one (1.2)

Chloromethyl methyl ether (10.29 g, 9.708 mL, 2.2 Eq, 127.8 mmol) was added drop-wise within 5 min to a solution of 1-(2,3,4-trihydroxyphenyl)ethan-1-one (9.769 g, 1 Eq, 58.10 mmol), DIPEA (30.04 g, 40.5 mL, 4 Eq, 232.4 mmol) in DCM (225 mL) at 0° C. The reaction mixture was stirred for 2 h at 0° C. before it was allowed to warm to room temperature. The reaction mixture was diluted with 500 ml of DCM, washed with 2×125 ml of 10% citric acid and 200 ml of brine. Organic layer was then dried with sodium sulfate, filtered and evaporated to dryness. Crude product was purified by normal phase flash-chromatography using EtOAc:heptane as the eluent system. 1-(2-Hydroxy-3,4-bis(methoxymethoxy)phenyl)ethan-1-one (12.617 g, 49 mmol, 84%, 99% Purity) was obtained as a pale yellow oil.

### (E)-1-(2-Hydroxy-3,4-bis(methoxymethoxy)phenyl)-3-(4-(3-((tetrahydro-2H-pyran-2-yl)oxy)propyl)phenyl)prop-2-en-1-one (12.4)

Sodium methoxide (4.1 g, 14 mL, 5.4 molar, 36 Eq, 76 mmol) in MeOH was added portion-wise under nitrogen flow to an ice/water cooled (0° C.) solution of 1-(2-hydroxy-3,4-bis(methoxymethoxy)phenyl)ethan-1-one (1.2, 0.544 g, 1 Eq, 2.124 mmol) and 4-(3-((tetrahydro-2H-pyran-2-yl)oxy)propyl)benzaldehyde (12.3, 0.554 g, 1.05 Eq, 2.23 mmol) in 1,4-dioxane (8 mL). The mixture was allowed slowly to warm to room temperature and it was stirred for 15 h under nitrogen atmosphere. The reaction mixture was concentrated, until most of MeOH was evaporated, and poured to 75 ml of ice-cold brine. The aqueous layer diluted with 15 ml of water and extracted with 3×75 ml of EtOAc. Organic layers were combined, dried with sodium sulfate, filtered through paper filter and evaporated to dryness. Crude product was purified by normal phase flash-chromatography using EtOAc:heptane as the eluent system. (E)-1-(2-Hydroxy-3,4-bis(methoxymethoxy)phenyl)-3-(4-(3-((tetrahydro-2H-pyran-2-yl)oxy)propyl)phenyl-)prop-2-en-1-one (0.8957 g, 1.6 mmol, 77%, 89% Purity) was obtained as an orange oil.

### 7,8-Dihydroxy-2-(4-(3-hydroxypropyl)phenyl)-4H -chromen-4-one (12.5)

Solution of (E)-1-(2-hydroxy-3,4-bis(methoxymethoxy)phenyl)-3-(4-(3-((tetrahydro-2H-pyran-2-yl)oxy)propyl)phenyl)prop-2-en-1-one (12.4) (1.996 g, 1 Eq, 4.102 mmol) and iodine (114 mg, 0.109 Eq, 449 μmol) in DMSO (30 mL) was heated in nitrogen atmosphere at 120° C. for 17 h. Reaction mixture was allowed to cool to room temperature before it was poured to 300 mL of 5% of Na₂SO₃ and the mixture was acidified with c. HCl until pH=2-3, filtered and washed with water. The resultant filter cake was dissolved in MeOH and dried. 7,8-Dihydroxy-2-(4-(3-hydroxypropyl)phenyl)-4H-chromen-4-one (1.22 g, 2.9 mmol, 71%, 75% purity) was obtained as a dark brown solid.

### 2-(4-(3-Bromopropyl)phenyl)-7,8-dihydroxy-4H-chromen-4-one (12.6).

1H-Benzo[d][1,2,3]triazole (254 mg, 1.3 Eq, 2.14 mmol) and N,N-dimethylformamide (2 6.4 mg, 27.9 μL, 0.22 Eq, 361 μmol) were added to a suspension of 7,8-dihydroxy-2-(4-(3-hydroxypropyl)phenyl)-4H-chromen-4-one (12.5, 0.513 g, 1.0 Eq, 1.64 mmol) in dry DCM (18 mL) under nitrogen flow at 0° C. The reaction mixture was slowly allowed to warm to room temperature and it was stirred at room temperature for 16 h, before it was cooled with an ice-bath and quenched with 20 mL of sat. NaHCO₃. The resultant suspension was extracted with 8×25 mL of DCM. Organic layers were combined and washed with 75 mL of brine, which in turn was extracted with 3×75 mL of DCM. Organic layers were combined, evaporated to dryness and purified by normal phase flash-chromatography using DCM:MeOH as the eluent system. 2-(4-(3-Bromopropyl)phenyl)-7,8-dihydroxy-4H-chromen-4-one (0.233 g, 0.56 mmol, 34%, 90% purity) was obtained as a brown powder.

### 7,8-Dihydroxy-2-(4-(3-(4-methylpiperazin-1-yl)propyl)phenyl)-4H-chromen-4-one dihydrochloride (12B/SND170)

A slurry of 2-(4-(3-bromopropyl)phenyl)-7,8-dihydroxy-4H-chromen-4-one (12.6) (0.201 g, 1 Eq, 536 μmol) in MeCN (6 mL) was added to a mixture of DIPEA (0.11 g, 0.15 mL, 1.6 Eq, 0.86 mmol), 1-methylpiperazine (0.3 g, 0.3 mL, 5 Eq, 3 mmol) and MeCN (0.5 mL). Mixture was stirred at room temperature for 70 h, before it was evaporated to dryness and dissolved in 20 mL of DCM. The resultant suspension was washed with 50 mL of brine:water 1:1 mixture. Aqueous layer was extracted with 6×50 mL of DCM and 7×50 mL of DCM:MeOH 9:1 mixture. Organic layers were combined, dried with sodium sulfate, filtered and evaporated to dryness. Crude product was purified by reversed-phase chromatography to yield 7,8-dihydroxy-2-(4-(3-(4-methylpiperazin-1-yepropyl)phenyl)-4H-chromen-4-one dihydrochloride (65 mg, 0.13 mmol, 25%, 95% purity) as an orange powder.

### 2-(3-(4-Bromophenyl)propoxy)tetrahydro-2H-pyran (12.2)

A solution of 3-(4-bromophenyl)propan-1-ol (12.1) (24.97 g, 1 Eq, 116.1 mmol) in dichloromethane (250 mL) was cooled under gentle nitrogen flow to 0° C. in a 500 ml round-bottom flask. p-Toluenesulfonic acid monohydrate (2.21 g, 0.111 Eq, 12.8 mmol) was then added portion-wise. 3,4-Dihydro-2H-pyran (19.35 g, 1.981 Eq, 230.0 mmol) was added drop-wise from a dropping funnel within 30 min before the mixture was allowed to warm to room-temperature. The solution turned eventually to black. The reaction mixture was stirred at room temperature for 16 hours before it was concentrated. The resultant black oil was purified by flash-chromatography using ethyl acetate/heptanes to yield 2-(3-(4-bromophenyl)propoxy)tetrahydro-2H-pyran (12.2) (28.8 g, 96.3 mmol, 83%, 100% purity) as a transparent oil.

### 4-(3-((Tetrahydro-2H-pyran-2-yl)oxy)propyl)benzaldehyde (12.3)

2-(3-(4-Bromophenyl)propoxy)tetrahydro-2H-pyran (12.2, 27.67 g, 1 Eq, 92.48 mmol) and THF (310 mL) were transferred under nitrogen flow to a flame-dried 500 ml three-neck round-bottom flask. The solution was cooled under gentle nitrogen flow to −75° C., before n-butyllithium (6.49 g, 40.5 mL, 2.5 molar, 1.09 Eq, 101 mmol) in hexanes was added portion-wise within 20 min. After 30 min of stirring, dry DMF was added portion-wise within 25 min and the reaction mixture was stirred for another 5 min before the cooling bath was removed. The reaction mixture was then stirred at 20° C. for 2 hour, before the reaction mixture was quenched with 100 ml of water and diluted with 900 ml of water. Resulting suspension was extracted with 3×750 ml of EtOAc. The organic fractions were combined, dried with sodium sulfate, filtered and concentrated to give the crude product as a yellow oil. The crude product was purified byflash-chromatography using ethyl acetate/heptanes to yield 4-(3-((tetrahydro-2H-pyran-2-yl)oxy)propyl)benzaldehyde (12.3) (18.7 g, 75 mmol, 81%, 99% purity) as a colorless oil.

### 1-(4-Hydroxy-2,2-diphenylbenzo[d][1,3]dioxol-5-yl)ethan-1-one (12.7)

1-(2,3,4-Trihydroxyphenyl)ethan-1-one (10.86 g, 1 Eq, 64.59 mmol), dichlorodiphenylmethane (15.29 g, 12.38 mL, 1.00 Eq, 64.48 mmol) and diphenyl ether (85 mL) were transferred under nitrogen flow to a 250 ml three-neck flask. The reaction mixture was heated at 175° C. for 30 min. The reaction mixture was allowed to cool to room temperature before it was poured to 900 ml of heptane. After a couple of minutes, precipitate started to form. This was filtered and washed with heptane. The dark precipitate on the filter was dissolved in DCM, 25 mL of EtOAc and 25 mL of heptane was added. This mixture was then concentrated until extensive precipitate formed. This was filtered, washed with 4×25 mL of EtOAc:heptane 1:1 mixture and purified by normal phase flash-chromatography using EtOAc:heptane as the eluent. The filtrate of the first filtration was concentrated, cooled to 4° C. for 20 h, filtered and washed with heptane This was combined with the material recovered from flash-chromatography to yield 1-(4-hydroxy-2,2-diphenylbenzo[d][1,3]dioxol-5-yl)ethan-1-one (12.7) (15.62 g, 47.0 mmol, 73%, 100% purity) as a white solid.

### (E)-1-(4-Hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8a) and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b)

Sodium methoxide (37.9 g, 130 mL, 5.4 molar, 35.7 Eq, 702 mmol) in MeOH was added portion-wise under nitrogen flow to an ice/NaCl cooled suspension of 1-(4-hydroxy-2,2-diphenylbenzo[d][1,3]dioxol-5-yl)ethan-1-one (6.5287 g, 1 Eq, 19.643 mmol) and 4-(3-((tetrahydro-2H-pyran-2-yl)oxy)propyl)benzaldehyde (5.037 g, 1.033 Eq, 20.28 mmol) in 1,4-dioxane (70 mL) at 0° C. The mixture was allowed slowly to warm to room temperature and it was stirred for 15 h under nitrogen atmosphere. The reaction mixture was then poured to 500 ml of ice-cold brine. The resultant suspension was extracted with 3×100 ml of EtOAc. Organic fractions were combined, dried with sodium sulfate, filtered and evaporated to dryness, yielding 14.27 g of dark orange oil. The crude product was suspended in DCM and purified twice by normal phase flash-chromatography using DCM:MeOH as the eluent, to yield (E)-1-(4-hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8a) (5.16 g, 9.17 mmol, 46.7%, 100% purity) as an orange foam and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b) (4.825 g, 7.7 mmol, 39%, 90% purity) a yellow foam.

### 2,2-Diphenyl-8-(4-(3-((tetrahydro-2H-pyran-2-yl)oxy)propyl)phenyl)-7,8-dihydro-6H-[1,3]dioxolo[4,5-h]chromen-6-one (12.9)

A solution of 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b) (4.825 g, 1 Eq, 8.575 mmol) and diiodine (223 mg, 0.102 Eq, 879 μmol) in DMSO (60 mL) was heated at 120° C. for 17 h. Reaction mixture was then allowed to cool to room temperature before it was poured to 600 ml of 1% sodium sulfite solution. Brown precipitate formed. The organic layer was extracted with 3×250 ml of EtOAc. Brine was added to speed up the separation of layers. Organic layers were combined and washed with 200 ml of brine, which in turn was extracted with 100 ml of EtOAc. Organic layers were combined, dried with sodium sulfate, filtered and evaporated to dryness to yield 3.93 g of dark oil. Crude product was suspended in DCM and purified by normal phaseflash-chromatography. 8-(4-(3-Hydroxypropyl)phenyl)-2,2-diphenyl-6H-[1,3]dioxolo[4,5-h]chromen-6-one (12.9) (2.151 g, 4.51 mmol, 52.6%, 100% purity) was obtained as a pale yellow solid.

### 8-(4-(3-Bromopropyl)phenyl)-2,2-diphenyl-6H-[1,3]dioxolo[4,5-h]chromen-6-one (12.10)

8-(4-(3-Hydroxypropyl)phenyl)-2,2-diphenyl-6H-[1,3]dioxolo[4,5-h]chromen-6-one (12.9, 2.15 g, 1 Eq, 4.51 mmol) was dissolved in dry DCM (36 mL) and was cooled to 0° C. under nitrogen atmosphere. N,N-dimethylformamide (0.9 g, 0.9 mL, 3 Eq, 0.01 mol) was then added under nitrogen flow, followed by sulfurous dibromide (1.2 g, 0.45 mL, 1.3 Eq, 5.8 mmol). After a few minutes, the cooling bath was removed and the orange solution was stirred at 20° C. Reaction was followed by LC-MS. After 105 min, the reaction mixture was cooled with ice-bath and 50 ml of sat. NaHCO₃ was added. The mixture was then extracted with 3×100 ml of DCM, until last fraction had very little UV-activity. Organic layers were combined, washed with 150 ml of brine, which in turn was extracted with 2×50 ml of DCM and dried with sodium sulfate. The solution was filtered, evaporated to dryness and purified by normal phaseflash-chromatography, using EtOAc:heptane as the eluent. 8-(4-(3-Bromopropyephenyl)-2,2-diphenyl-6H-[1,3]dioxolo[4,5-h]chromen-6-one (2.014 g, 3.73 mmol, 82.8%, 100% purity) was obtained as a white solid.

### General Method for Alkylation of Secondary Amines with 8-(4-(3-bromopropyl)phenyl)-2,2-diphenyl-6H-[1,3]dioxolo[4,5-h]chromen-6-one (12.10)

1-5 eq. of the secondary amine with 1.5 eq. of DiPEA in MeCN was added to a suspension of 8-(4-(3-bromopropyephenye-2,2-diphenyl-6H-[1,3]dioxolo[4,5-h]chromen-6-one (12.10) in MeCN. Reaction mixture was stirred at room temperature or at 50° C. under nitrogen atmosphere until full conversion was achieved by TLC or LC-MS. Reaction mixture was then concentrated, dissolved in DCM and washed with brine:water 1:1 mixture. The aqueous layer was extracted twice with DCM. Organic layers were combined, dried with sodium sulfate, filtered and evaporated to dryness. Crude product was purified by normal phase flash-chromatography using DCM:NH₃ in MeOH as the eluent.

### 8-(4-(3-Morpholinopropyl)phenyl)-2,2-diphenyl-6H-[1,3]dioxolo[4,5-h]chromen-6-one (12.11A)

8-(4-(3-Bromopropyephenyl)-2,2-diphenyl-6H-[1,3]dioxolo[4,5-h]chromen-6-one (12.10) (0.225 g, 1 Eq, 417 μmol) was treated with morpholine (0.2 g, 0.21 mL, 6 Eq, 2 mmol) and DIPEA (80.9 mg, 109 μL, 1.50 Eq, 626 μmol) in MeCN (3 mL) at room temperature for 18 h, followed by 7.5 h at 50° C. After work-up and purification, 8-(4-(3-morpholinopropyl)phenyl)-2,2-diphenyl-6H-[1,3]dioxolo[4,5-h]chromen-6-one (0.218 g, 400 μmol, 95.8%, 100% purity) as an off-white foam.

### 2,2-Diphenyl-8-(4-(3-(piperidin-1-yl)propyl)phenyl)-6H-[1,3]dioxolo[4,5-h]chromen-6-one (12.11C)

Suspension of 8-(4-(3-bromopropyl)phenyl)-2,2-diphenyl-6H-[1,3]dioxolo[4,5-h]chromen-6-one (12.10) (0.273 g, 1 Eq, 506 μmol) in MeCN (2 mL) was treated with piperidine (215 mg, 250 μL, 5 Eq, 2.53 mmol) and DIPEA (98.1 mg, 132 μL, 1.5 Eq, 759 μmol) in MeCN (2 mL) at room temperature for 18 h. After work-up and purification, 2,2-diphenyl-8-(4-(3-(piperidin-1-yl)propyl)phenyl)-6H -[1,3]dioxolo[4,5-h]chromen-6-one (0.2327 g, 428 μmol, 84.6%, 100% purity) was obtained as a pale yellow foam.

### 8-(4-(3-(Methyl(2-(piperidin-1-yl)ethyl)amino)propyl)phenyl)-2,2-diphenyl-6H-[1,3]dioxolo[4,5-h]chromen-6-one (12.11E)

Suspension of 8-(4-(3-bromopropyephenyl)-2,2-diphenyl-6H-[1,3]dioxolo[4,5-h]chromen-6-one (12.10) (0.549 g, 1 Eq, 1.02 mmol) in MeCN (4 mL) was treated with N-methyl-2-(piperidin-1-yl)ethan-1-amine (306 mg, 2.11 Eq, 2.15 mmol) and DIPEA (197 mg, 266 μL, 1.50 Eq, 1.53 mmol) in MeCN (1 mL) at room temperature for 17 h and at 50° C. for 22 h. After work-up and purification, 8-(4-(3-(methyl(2-(piperidin-1-yl)ethyl)amino)propyephenyl)-2,2-diphenyl-6H-[1,3]dioxolo[4,5-h]chromen-6-one (0.584 g, 0.94 mmol, 93%, 97% purity) was obtained as an orange oil.

### 8-(4-(3-(Methyl(2-(4-methylpiperazin-1-yl)ethyl)amino)propyl)phenyl)-2,2-diphenyl-6H-[1,3]dioxolo[4,5-h]chromen-6-one (12.11F).

Suspension of 8-(4-(3-bromopropyephenyl)-2,2-diphenyl-6H-[1,3]dioxolo[4,5-h]chromen-6-one (12.10) (0.229 g, 1 Eq, 425 μmol) in MeCN (1 mL) was treated with N-methyl-2-(4-methylpiperazin-1-yl)ethan-1-amine (177 mg, 2.65 Eq, 1.13 mmol) and DIPEA (82.3 mg, 111 μL, 1.5 Eq, 637 μmol) in MeCN (2 mL) at 50° C. for 20 h. After work-up and purification, 8-(4-(3-(methyl(2-(4-methylpiperazin-1-yl)ethyl)amino)propyl)phenyl)-2,2-diphenyl-6H-[1,3]dioxolo[4,5-h]chromen-6-one (0.2406 g, 0.35 mmol, 83%, 90% purity) was obtained as a slightly yellow oil.

### tert-Butyl-(4-((tert-butoxycarbonyl)amino)butyl) (3-((3-(4-(6-oxo-2,2-diphenyl-6H-[1,3]dioxolo[4,5-h]chromen-8-yl)phenyl)propyl)amino)propyl)carbamate (12.11G).

Suspension of 8-(4-(3-bromopropyl)phenyl)-2,2-diphenyl-6H-[1,3]dioxolo[4,5-h]chromen-6-one (12.10) (0.2076 g, 1 Eq, 384.9 μmol) in MeCN (1 mL) was treated with tert-butyl-(3-aminopropyl)(4-((tert-butoxycarbonyl)amino)butyl)carbamate (162 mg, 1.22 Eq, 469 μmol) and DIPEA (74.9 mg, 101 μL, 1.51 Eq, 580 μmol) in MeCN (2 mL) at 50° C. for 3 h, followed by 60° C. for 18 h and 90° C. for 24 h. After work-up and purification, tert-butyl-(4-((tert-butoxycarbonyl)amino)butyl)(3-((3-(4-(6-oxo-2,2-diphenyl-6H-[1,3]dioxolo[4,5-h]chromen-8-yl)phenyl)propyl)amino)propyl)carbamate (0.179 g, 0.21 mmol, 54%, 94% purity) was obtained as a yellow foam.

### tert-Butyl-(3-((tert-butoxycarbonyl)amino)propyl)(4-((3-(4-(6-oxo-2,2-diphenyl-6H-[1,3]dioxolo[4,5-h]chromen-8-yl)phenyl)propyl)amino)butyl)carbamate (12.11H).

Suspension of 8-(4-(3-bromopropyl)phenyl)-2,2-diphenyl-6H-[1,3]dioxolo[4,5-h]chromen-6-one (12.10) (0.2108 g, 1 Eq, 390.8 μmol), potassium carbonate (170 mg, 3.15 Eq, 1.23 mmol) and potassium iodide (5 mg, 0.08 Eq, 0.03 mmol) in MeCN (1 mL) was treated with tert-butyl-(4-aminobutyl)(3-((tert-butoxycarbonyl)amino)propyl)carbamate (176 mg, 1.30 Eq, 509 μmol) in MeCN (2 mL). The mixture was then heated to 90° C. for 20 h. After work-up and purification, tert-butyl (3-((tert-butoxycarbonyl)amino)propyl)(4-((3-(4-(6-oxo-2,2-diphenyl-6H-[1,3]dioxolo[4,5-h]chromen-8-yl)phenyl)propyl)amino)butyl)carbamate (163 mg, 0.19 mmol, 50%, 96% purity) was obtained as a slightly yellow powder.

### 1,3-Bis-tert-butoxycarbonyl-1-(3-(4-(6-oxo-2,2-diphenyl-6H-[1,3]dioxolo[4,5-h]chromen-8-yl)phenyl)propyl)guanidine (12.11J).

Solution of 8-(4-(3-bromopropyl)phenyl)-2,2-diphenyl-6H-[1,3]dioxolo[4,5-h]chromen-6-one (12.10) (255 mg, 1 Eq, 473 μmol) in DMF (3.5 mL) was treated with 1,3-bis(tert-butoxycarbonyl)guanidine (252 mg, 2.06 Eq, 972 μmol), potassium carbonate (132 mg, 2.02 Eq, 955 μmol) and potassium iodide (14 mg, 0.18 Eq, 84 μmol) in DMF (1.5 mL) at 50° C. for 18 h. After work-up and purification, 1,3-bis-tert-butoxycarbonyl-1-(3-(4-(6-oxo-2,2-diphenyl-6H-[1,3]dioxolo[4,5-h]chromen-8-yl)phenyl)propyl)guanidine (0.302 g, 0.40 mmol, 85%, 95% purity) was obtained as a white solid.

### 7,8-Dihydroxy-2-(4-(3-(methyl(2-(piperidin-1-yl)ethyl)amino)propyl)phenyl)-4H-chromen-4-one dihydrochloride (12E/SND171).

8-(4-(3-(Methyl(2-(piperidin-1-yl)ethyl)amino)propyl)phenyl)-2,2-diphenyl-6H-[1,3]dioxolo[4,5-h]chromen-6-one (12.11E) (0.555 g, 1.00 Eq, 924 μmol) was dissolved in MeOH (5 mL), 23 mL of 4 M HCl in 1,4-dioxane (3.37 g, 100 Eq, 92.4 mmol) was added and the mixture was stirred at room temperature for 18 h. The reaction mixture was concentrated, diluted with 50 mL of Et₂O and filtered. The solid on the filter was then purified by reversed-phase chromatography to yield 7,8-dihydroxy-2-(4-(3-(methyl(2-(piperidin-1-yl)ethyl)amino)propyl)phenyl)-4H-chromen-4-one dihydrochloride (0.356 g, 699 μmol, 75.6%, 97% purity) an orange solid.

### 7,8-Dihydroxy-2-(4-(3-(methyl(2-(4-methylpiperazin-1-yl)ethyl)amino)propyl)phenyl)-4H-chromen-4-one trihydrochloride (12F/SND173).

8-(4-(3-Methyl(2-(4-methylpiperazin-1-yl)ethyl)amino)propyl)phenyl)-2,2-diphenyl-6H-[1,3]dioxolo[4,5-h]chromen-6-one (12.11F) was dissolved in MeCN (2 mL) and deprotected with c. HCl (956 mg, 808 μL, 37% Wt, 25 Eq, 9.70 mmol). After concentration, filtration, washing and drying, 7,8-dihydroxy-2-(4-(3-methyl(2-(4-methylpiperazin-1-yl)ethyl)amino)propyl)phenyl)-4H-chromen-4-one trihydrochloride (189 mg, 0.31 mmol, 81%, 93% purity) was obtained as an orange powder.

### 2-(4-(3-((3-((4-Aminobutyl)amino)propyl)amino)propyl)phenyl)-7,8-dihydroxy-4H-chromen-4-one trihydrochloride (12G/SND175).

tert-Butyl-(4-((tert-butoxycarbonyl)amino)butyl)(3-((3-(4-(6-oxo-2,2-diphenyl-6H-[1,3]dioxolo[4,5-h]chromen-8-yl)phenyl)propyl)amino)propyl)carbamate (12.11G) (0.189 g, 1 Eq, 235 μmol) was dissolved in MeCN (1.5 mL) and deprotected with c. HCl (0.59 g, 0.50 mL, 37% Wt, 25 Eq, 6.0 mmol). After concentration, filtration, washing and drying, 2-(4-(3-((3-((4-aminobutyl)amino)propyl)amino)propyl)phenyl)-7,8-dihydroxy-4H-chromen-4-one trihydrochloride (103 mg, 188 μmol, 79.8%, 95% purity) was obtained as a yellow powder.

### 2-(4-(3-((4-((3-Aminopropyl)amino)butyl)amino)propyl)phenyl)-7,8-dihydroxy-4H-chromen-4-one trihydrochloride (12H/SND174).

tert-Butyl(3-((tert-butoxycarbonyl)amino)propyl)(4-((3-(4-(6-oxo-2,2-diphenyl-6H-[1,3]dioxolo[4,5-h]chromen-8-yl)phenyl)propyl)amino)butyl)carbamate (12.11H) (163 mg, 1 Eq, 203 μmol) was dissolved in MeCN (1 mL) and deprotected with c. HCl (0.35 g, 0.30 mL, 37% Wt, 18 Eq, 3.6 mmol). After concentration, filtration, washing and drying, 2-(4-(3-((4-((3-aminopropyl)amino)butyl)amino)propyl)phenyl)-7,8-dihydroxy-4H-chromen-4-one trihydrochloride (87 mg, 0.16 mmol, 78%, 97% purity) was obtained as a pale yellow powder.

### 1-(3-(4-(7,8-Dihydroxy-4-oxo-4H-chromen-2-yl)phenyl)propyl)guanidine hydrochloride (12J/SND177).

1,3-Bis-tert-butoxycarbonyl-1-(3-(4-(6-oxo-2,2-diphenyl-6H-[1,3]dioxolo[4,5-h]chromen-8-yl)phenyl)propyl)guanidine (12.11J) was suspended in MeCN (2 mL) and deprotected with c. HCl (1.19 g, 1.01 mL, 37% Wt, 25 Eq, 12.1 mmol). After concentration, filtration, washing and drying, 1-(3-(4-(7,8-dihydroxy-4-oxo-4H-chromen-2-yl)phenyl)propyl)guanidine hydrochloride (177 mg, 0.44 mmol, 90%, 96% purity) was obtained as an orange powder.

Other compounds falling within the scope of the claims can be synthesized using the similar procedures.

### EXAMPLES—BIOLOGICAL STUDIES

### Experimental Methodology

### Antitumor Activity Against a Panel of Cancer Cell Lines

Antitumor activity of the compounds and doxorubicin as a positive control was assessed by using the CellTiter-Blue Cell Viability Assay (Promega, #G8082) or CellTiter-Glow® Luminescent Cell Viability assay (Promega # G7572) according to the manufacturer's instructions. The compounds were tested at 5 or 6 concentrations in half-log increments (highest concentration 30 μM-100 μM) in duplicate or triplicate well conditions.

Tumor cells were grown at 37° C. in a humidified atmosphere with 5% CO₂ in RPMI 1640 or DMEM medium, supplemented with 10% (v/v) fetal calf serum and 50 μg/ml gentamicin for up to 20 passages, and were passaged once or twice weekly. Cells were harvested using TrypLE or PBS buffer containing 1 mM EDTA, and the percentage of viable cells is determined using a CASY Model TT cell counter (OMNI Life Science). Cells were harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 or DMEM medium supplemented with 10% (v/v) fetal calf serum and 50 μg/ml gentamicin (140 μl/well). Cultures were incubated at 37° C. and 5% CO₂ in a humidified atmosphere. After 24 h, 10 μl of test compounds or control medium are added, and left on the cells for another 72 h. Compounds were serially diluted in DMSO, transferred in cell culture medium, and added to the assay plates. The DMSO concentration was kept constant at <0.3% v/v across the assay plate. Viability of cells was quantified by the CellTiter-Blue® cell viability assay (Promega G8081) or CellTiter-Glow® Luminescent Cell Viability assay (Promega # G7572). Fluorescence (FU) was measured by using the EnSpire® multimode plate reader (Perkin Elmer) (excitation λ=570 nm, emission λ=600 nm) or luminescent detection of ATP in viable cells was used as parameter for proliferation. Luminescence was measured with the microplate luminometer (Promega or Perkin Elmer).

Sigmoidal concentration-response curves were fitted to the data points (test-versus-control, T/C values) obtained for each tumor model using 4 parameter non-linear curve fit (Charles River DRS Datawarehouse Software) or with GraphPad prism 5.02 software. IC₅₀ values are reported as absolute IC₅₀ values, being the concentration of test compound at the intersection of the concentration-response curves with T/C=50%

Cell lines tested are presented in Table 1.

TABLE 1
Tumour cell lines type and designation

| Tumour model | Cell line |
|---|---|
| Brain | U-87MG |
| | IMR-5-75 |
| | SK-N-SH |
| | Kelly |
| | SH-SY-5Y |
| | LN-229 |
| Breast | MCF-7 |
| | MDA-MB-468 |
| Colon | HC-T116 |
| Leukemia | K-562 |
| | HL-60 |
| | MOLM-16 |
| | NALM-6 |
| Lung | A-549 |
| (NSCLC) | H-1299 |
| | Calu-6 |
| | NCI-H1437 |
| | NCI-H460 |
| Lung | H69AR |
| (SCLC) | NCI-H69 |
| | DMS-114 |
| Lung | NCI-H292 |
| | NCI-H441 |
| Lymphoma | GA-10 |
| | NAMALWA |
| | SU-DHL-4 |
| | H9 |
| | DOHH-2 |
| Ovarian | SK-OV-3 |
| Pancreatic | Mia-Pa-Ca-2 |
| | Panc-1 |
| Renal | 486L |
| Skin | A375 |
| (Melanoma) | MeWo |

### Antitumor Activity Against a Panel of Patient-Derived Xenografts (PDX)

PDX-derived cell cultures were obtained from tumors explanted from mice and isolated by mechanical and enzymatic dissociation. Assays were performed on cells from frozen stocks at least 2 weeks after thawing and maintained in culture at 37° C. in a humidified atmosphere with 5% CO2 in complete growth medium supplemented with 8 to 16% fetal bovine serum, 1% Penicillin-Streptomycin (10,000 U/mL), 2 mM L-Glutamine +/−Insulin-Transferrin-Selenium 1× and Albumax II (10 to 40 μM depending on cell type). Cells were harvested and seeded in 96-wells plates at a density of 1.25 to 5×10³ cells/well for cytotoxicity assays. Cells were incubated 48 h at 37° C. prior to addition of test molecules and vehicle (DMSO, 0.1%) at desired final concentrations. Cell viability was assessed before drugs' addition (T0) and 5 days after test molecules addition by measuring ATP cell content using CellTiter-Glo® Luminescent Cell Viability Assay (Promega) according to the manufacturer's instructions. Luciferase activity was measured on a luminometer (PerkinElmer® EnVision™). Each concentration of compounds was tested in triplicate.

Viability was calculated as a percentage of ATP value compared to vehicle treated controls.

For PDX primary cell cultures, the tumour tissue was washed with PBS containing antibiotic-antimycotic and non-tumour tissue and necrotic tumour tissues were separated. The tumour tissue was transferred to a new dish and cut into 1˜2 mm³ fragments, resuspended in RPMI-1640 medium and centrifuged at 1,200 rpm for 6 min at room temperature. The pelleted material was resuspended with 15 mL of Tumour Cell Digestion Solution and incubated at 37° C. for 1 hour with agitation. Following further addition of media, centrifugation and passage through a 70 mm cell strainer, the homogenous cell mixture was layered onto 15 mL of Ficoll-Paque PLUS in a 50 mL conical tube and centrifuged for 15 min at 1,600 rpm. The interface cells were collected, washed with media, separated by centrifugation at 1,200 rpm. The cell pellet was resuspended in serum free media supplemented with growth factors. 10,000 cells/wells were plated in a 96 well plate and incubated at 37° C., 5% CO₂, 95% air and 100% relative humidity overnight. The cytotoxicity assay was conducted as above. IC50 values represent absolute IC50.

PDX tested are presented in Table 2.

TABLE 2
PDX origin and designation

| Tumour organ | Model ID |
|---|---|
| Bile duct | CH-17-0091 |
| | CH-17-0098 |
| Brain | GBM14-CHA |
| | ODA14-RAV |
| Breast | HBCx-2 |
| | HBCx-3 |
| | HBCx-6 |
| | BR-05-0300 |
| | BR-05-0014E |
| Colon | TC71 |
| | CO-04-0722 |
| | CO-04-0701 |
| | CO-04-0700 |
| Esophagus | ES-06-0002 |
| | ES-06-0122 |
| Head and neck | HN-13-0020 |
| Kidney | Ki-12-0062 |
| Liver | HB-214-FOI |
| Lung | IC20-DAN |
| | SC6 |
| | LU-01-0027 |
| | LU-01-0010 |
| | LU-01-0604 |
| | LU-01-0025 |
| Lymphoma | LY-24-0304 |
| Pancreas | PC-07-0045 |
| | PC-07-0059 |
| Stomach | ST-02-0007 |
| | ST-02-0173 |
| | ST-02-0012 |
| | ST-02-0322 |

### Inhibition of Kinase Activity

Selected compounds were screened for kinase inhibition using the KINOMEscan™ assay (Eurofins) which is based on a competition binding assay that quantitatively measures the ability of a compound to compete with an immobilized, active-site directed ligand. The assay was performed by combining three components: DNA-tagged kinase; immobilized ligand; and the test compound. The ability of the test compound to compete with the immobilized ligand was measured via quantitative PCR of the DNA tag.

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 MM DTT) to remove unbound ligand and to reduce non-specific binding. Binding reactions were assembled by combining kinases, liganded affinity beads, and test compounds in 1× binding buffer (20% SeaBlock, 0.17× PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

Compounds were initially tested at a concentration of 10 mM against a panel of 30 kinases and results for primary screen binding interactions were reported as ‘% Ctrl’

% Ctrl Calculation=(test compound signal−positive control signal/negative control signal−positive control signal)×100, where negative control=DMSO (100% Ctrl) positive control=control compound (0% Ctrl). Test compounds with % Ctrl between 0 and 10 were selected for Kd determination.

Binding constants (Kds) were calculated with a standard dose-response curve using the Hill equation: Response=Background+[Signal−Background/1+(Kd^{Hill Slope}/Dose^{Hill Slope})]. The Hill Slope was set to −1. Curves were fitted using a non-linear least square fit with the Levenberg-Marquardt algorithm.

### In Vivo Activity Against Human Tumour Xenografts

In vivo activity against a number of CDX were evaluated in various nude or SCID mice. Tumour cells were inoculated subcutaneous (s.c) into the mice flank. Mice were randomized into groups when tumours were around 100-200 mm³. A vehicle control group (2.5-4% DMSO, 5% EtOH, 20% PEG200 in saline) was part of each experiment. Treatment was administered intraperitoneally (i.p.) 3 times a week or daily (qd).

Body weight was measured before each treatment and treatments of individual mice was paused, when the body weight loss was >15%. Tumor volumes were measured 2×/week. Mice were individually sacrificed, when the tumor volume reached the volume specificed in the laboratory internal SOP.

The tumour inhibition growth was assessed as the optimal T/C, where T represents the median tumour volume of the treated group and C representes the median tumour volume of the control (vehicle) group. Statistical analysis was performed using two way RM ANOVA with p values <0.05% considered statistically significant.

All procedures related to animal handling, care and the treatment in the study were performed according to the guidelines of the Association for Assessment and Accreditation of Laboratory Animal Care (AAALAC).

### Example 1. Activity Against Brain Carcinomas

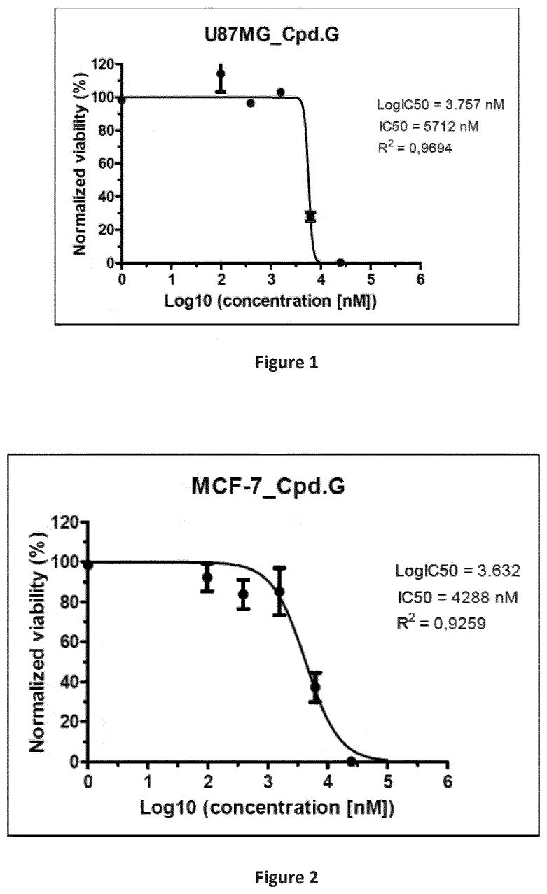

SND164, SND391, SND392, SND393, SND394 inhibited brain cancer cell growth with IC50s below 10 μM, as presented in Table 3A-D and FIG. 1.

TABLE 3A
IC50 values against brain carcinoma cell lines

| Cell Line/ | | | | | |
|---|---|---|---|---|---|
| IC50 (μM) | SND164 | SND391 | SND392 | SND393 | SND394 |
| U-87MG | 5.7 | 5.1 | 7.6 | 7.1 | 7.6 |

TABLE 3B
IC50 values against brain carcinoma cell lines

| Cell Line/ | | | | |
|---|---|---|---|---|
| IC50 (μM) | SND390 | SND391 | SND392 | SND394 |
| LN-229 | 7 | 2.8 | 11 | 2.9 |

TABLE 3C
IC50 values against brain carcinoma cell lines

| Cell Line/ | | | |
|---|---|---|---|
| IC50 (μM) | SND391 | SND392 | SND393 |
| SH-SY-5Y | 3 | 3.7 | 4.7 |

TABLE 3D
IC50 values against brain carcinoma cell lines

| Cell Line/ | | | | |
|---|---|---|---|---|
| IC50 (μM) | SND391 | SND392 | SND393 | SND394 |
| U-251 | 3.9 | 8.4 | 6.8 | 7.6 |

SND211 inhibited the growth of brain carcinoma cell line IMR-5-75 with an IC50 of 1.5 μM and SND214 inhibited the growth of brain carcinoma cell line SK-N-SH with an IC50 of 2.3 μM.

FIG. 1 shows IC50 curve of SND164 (Cpd G) activity against brain carcinoma.

### Example 2. Activity Against Breast Carcinomas

SND164, SND391, DND393 and SND394 inhibited breast cancer cell growth with IC50s below 10 μM, as presented in Table 4A and 4B and FIG. 2.

TABLE 4A
IC50 values against breast carcinoma cell lines

| Cell Line/ | |
|---|---|
| IC50 (μM) | SND164 |
| MCF-7 | 4.3 |

TABLE 4B
IC50 values against breast carcinoma cell lines

| Cell Line/ | | | |
|---|---|---|---|
| IC50 (μM) | SND391 | SND393 | SND394 |
| MDA-MB-468 | 1.2 | 3.2 | 3.8 |

FIG. 2 shows IC50 curve of SND164 (Cpd G) activity against breast carcinoma.

SND164 was active against breast PDX BR-05-0014E and HBCx-3 inhibiting tumour growth with IC50s of 13.3 μM and 10 μM respectively.

### Example 3. Activity Against Colon Carcinomas

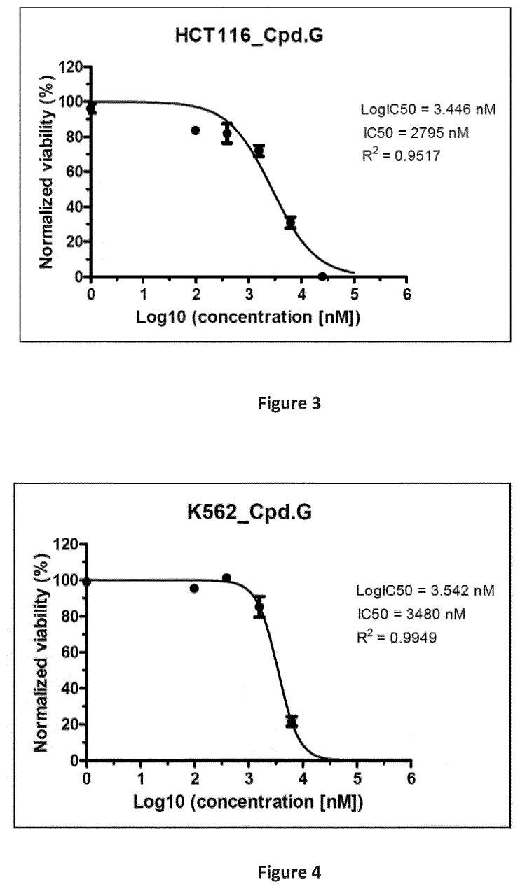

SND164 and SND172 inhibited colon cancer cell growth with IC50s below 20 μM, as presented in Table 5 and FIG. 3.

FIG. 3 shows IC50 curve of SND164 (Cpd G) activity against colon carcinoma.

TABLE 5
IC50 values against colon cancer cell line

| Cell Line/IC50 | | |
|---|---|---|
| (μM) | SND164 | SND172 |
| HCT-116 | 2.8 | 11.4 |

SND164 was active against colon PDX CO-04-0700 inhibiting tumour growth with an IC50 of 7 μM.

### Example 4. Activity Against Leukaemia

SND164 inhibited leukaemia cell growth with IC50 below 10 μM, as presented in Table 6A and FIG. 4.

TABLE 6A
IC50 values against leukaemia cell lines

| Cell Line/IC50 | |
|---|---|
| (μM) | SND164 |
| K562 | 3.48 |
| MOLT-16 | 4.36 |
| NALM-6 | 4.45 |

FIG. 4 shows IC50 curve of SND164 (Cpd G) activity against chronic myelogenous leukemia (CML).

The acute promyelocytic leukemia cell line HL-60 growth was inhibited by SND142, SND391, SND392 and SND394 as shown in Table 6B.

TABLE 6B
IC50 values against leukaemia cell lines

| Cell Line/IC50 | | | | |
|---|---|---|---|---|
| (μM) | SND142 | SND391 | SND392 | SND394 |
| HL-60 | 3.48 | 2.4 | 5.9 | 2.8 |

### Example 5. Activity Against Lung Carcinomas

SND142, SND146, SND147, SND SND164, SND167, SND168, SND211, SND214, SND391, SND392, SND393 and SND394 inhibited lung carcinoma cell growth with IC50s below 20 μM, as presented in Table 7A-E.

TABLE 7A
IC50 values against non-small cell lung carcinoma cell lines

| Cell | | | | | | |
|---|---|---|---|---|---|---|
| Line/IC50 | | | | | | |
| (μM) | SND142 | SND163 | SND164 | SND167 | SND168 | SND211 |
| NCI-H1299 | 1 | 5.8 | 1.25 | 2.7 | 3.7 | 1.8 |

TABLE 7B
IC50 values against non-small cell lung carcinoma cell lines

| Cell | | | | | | | |
|---|---|---|---|---|---|---|---|
| Line/IC50 | | | | | | | |
| (μM) | SND142 | SND146 | SND147 | SND391 | SND392 | SND393 | SND394 |
| CALU-6 | 1.7 | 17.18 | 12.19 | 4 | 7 | 7 | 8.9 |

TABLE 7C
IC50 values against non-small lung carcinoma cell line

| Cell Line/IC50 | | | | |
|---|---|---|---|---|
| (μM) | SND390 | SND391 | SND392 | SND394 |
| NCI-H1437 | 10 | 1.4 | 3.1 | 2.1 |

TABLE 7D
IC50 values against small cell lung carcinoma cell line

| Cell Line/IC50 | | | |
|---|---|---|---|
| (μM) | SND391 | SND392 | SND394 |
| DMS114 | 1.1 | 5.4 | 3.1 |

TABLE 7E
IC50 values against mucoepidermoid
pulmonary and adenocarcinoma

| Cell Line/IC50 | | | |
|---|---|---|---|
| (μM) | SND391 | SND392 | SND394 |
| NCI-H292 | 0.98 | 3.8 | 1.9 |
| NCI-H441 | 1.2 | 5.4 | 3.2 |

The multiple drug resistant cell line H69AR obtained from ATCC, was established from NCI-H69 cells that were grown in the presence of increasing concentrations of adriamycin (doxorubicin). The H69AR cell line is approximately 50-fold resistant to adriamycin as compared to the parental NCI-H69 cell line. SND141, SND142, SND143, SND146, SND147 and SND148 inhibited the resistant cell line H69AR with IC50s below 5 μM, as presented in Table 8.

TABLE 8
IC50 values against resistant small cell lung carcinoma cell line

| Cell | | | | | | |
|---|---|---|---|---|---|---|
| Line/IC50 | | | | | | |
| (μM) | SND141 | SND142 | SND143 | SND146 | SND147 | SND148 |
| H69AR | 2.0 | 1.2 | 3.3 | 2.2 | 2.6 | 2.5 |

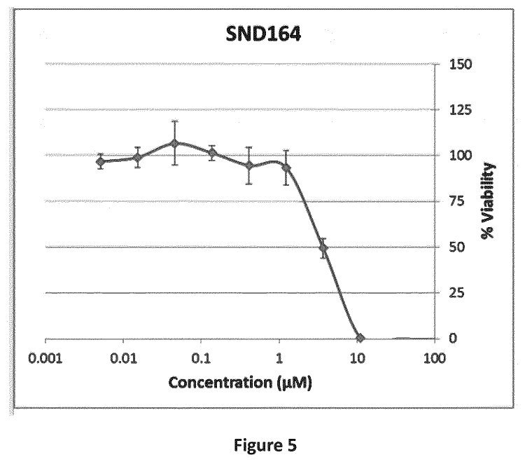

SND164 inhibited the PDX small cell lung carcinoma SC6 with an IC50 of 3.64 μM as shown in FIG. 5 and the lung carcinoma PDX LU-01-0025 with an IC50 of 5 μM. SND171 was also moderately active against SC6 with an IC50 of 15 μM.

FIG. 5 shows IC50 curve of SND164 (Cpd G) activity against small cell lung carcinoma PDX.

### Example 6. Activity Against Lymphomas

SND164 was particularly active against a series of lymphoma cell lines and PDX as depicted in Table 9 and 10.

TABLE 9
IC50 values against lymphoma cell llines

| Cell Line/IC50 | |
|---|---|
| (μM) | SND164 |
| NAWALMA | 2.6 |
| SU-DHL-4 | 3.3 |
| GA-10 | 3.9 |
| DOHH-2 | 4.3 |

TABLE 10
IC50 values against lymphoma PDX

| Cell Line/IC50 | |
|---|---|
| (μM) | SND164 |
| LY-24-0340 | <0.16 |

### Example 7. Activity Against Ovarian Carcinomas

SND211 inhibited ovarian carcinoma cell growth with IC50s below 20 μM, as presented in Table 11.

TABLE 11
IC50 values against ovarian carcinoma cell lines

| Cell Line/IC50 | |
|---|---|
| (μM) | SND211 |
| SK-OV-3 | 10.6 |

### Example 8. Activity Against Pancreatic Carcinomas

SND141, SND142, SND143, SND146, SND147 and SND148, inhibited pancreatic carcinoma cell growth with IC50s below 10 μM, as presented in Tables 12.

TABLE 12
IC50 values against pancreatic carcinoma cell lines

| Cell | | | | | | |
|---|---|---|---|---|---|---|
| Line/IC50 | | | | | | |
| (μM) | SND141 | SND142 | SND143 | SND146 | SND147 | SND148 |
| Panc-1 | 3.91 | 1.6 | 6.09 | 1.2 | 3.8 | 2.3 |
| MIA-Pa-Ca-2 | 2.3 | 1 | 4.96 | 0.87 | 2.02 | 2.21 |

### Example 9. Activity Against Renal Carcinomas

SND143, SND 146, SND147, SND148, inhibited multi drug resistant (MDR) renal patient derived line RXF486 growth with IC50s below 2 μM, as presented in Table 13.

TABLE 13
IC50 values against MDR renal carcinoma cell lines

| Cell Line/IC50 | | | | |
|---|---|---|---|---|
| (μM) | SND143 | SND146 | SND147 | SND148 |
| RXF 486 | 1.3 | 0.4 | 0.6 | 0.6 |

### Example 10. Activity Against Skin Cancer (Melanoma)

SND173, SND211, SND391, SND392 and SND393 inhibited melanoma cell lines growth with IC50s below 10 μM as presented in Table 14A and 14B.

TABLE 14A
IC50 values against melanoma cell lines

| Cell Line/IC50 | | | |
|---|---|---|---|
| (μM) | SND211 | SND391 | SND392 |
| MeWo | 5.3 | 1.9 | 9.8 |

TABLE 14B
IC50 values against melanoma cell lines

| Cell Line/IC50 | | | | |
|---|---|---|---|---|
| (μM) | SND173 | SND211 | SND391 | SND393 |
| A375 | 10 | 1.8 | 1.3 | 2.7 |

### Example 11. Activity Against Other Solid Tumour PDX

SND148 inhibited the bile duct PDX CH-17-0098 growth with an IC50 of 13.9 μM, whereas SND164 was active against CH-17-0091 with an IC50 of 13.8 μM.

SND164 inhibited the head and neck PDX HN-13-0020, the esophagus PDX ES-06-0122 and stomach ST-02-0322 growth as shown in Table 15.

TABLE 15
IC50 values against various PDX

| Cell Line/IC50 | |
|---|---|
| (μM) | SND164 |
| HN-13-0020 | 2.7 |
| ES-06-0122 | 5.7 |
| ST-02-0322 | 5.45 |

### Example 12. Kinase Inhibition Activity

In order to further understand if the tumour inhibition activity is due to the inhibition of certain cancer associated kinases, selected compounds were tested in the KINOMEscan™ assay against 30 kinases. SND164 showed selective inhibitory activity against a small number of kinases as presented in Table 16.

TABLE 16
Kd values kinase inhibition

| Kinase | Kd (μM) |
|---|---|
| ABL1(E255K)-phosphorylated | 0.68 |
| ABL1-phosphorylated | 0.77 |
| PIM1 | 0.58 |
| PIM2 | 0.63 |
| PIM3 | 0.43 |
| RSK2(Kin.Dom.1-N-terminal) | 1.4 |
| SNARK | 0.9 |

### Example 13. In Vivo Maximum Tolerated Dose

In order to evaluate the general toxicity of selected SND compounds in an animal model, a maximum tolerated dose (MTD) in NOG mice has been undertaken. Three mice per group were treated daily or every other day over 8 days at various doses between 5 mg/kg and 10 mg/kg. Administration of the compounds was i.p. A stock solution of the compounds (40 mg/ml in DMSO) was prepared and frozen at −20° C. in aliquots for each treatment. The compounds were freshly diluted with vehicle (2.5% DMSO/5% EtOH/20% PEG200/72.5% saline) before the treatments. Body weights were measured before each treatment. Mice were further observed for 1 week after the last treatment.

Compounds SND142, SND143, SND147, SND148, and SND164 were well tolerated at the highest dose administered in these studies, 8-10 mg/kg with body weight of the animals remaining below 5% and no abnormalities detected on autopsy. While the MTD was not reached in these studies, based on the data it is estimated at >10 mg/kg upon repeated i.p. administration.

### Example 14. In Vivo Tumour Inhibition of Xenograft Human Glioblastoma

Selected test compounds were assessed for their potential to inhibit human xenografts growth in vivo. To this end, 1×10⁷ U87MG human glioblastoma cells were transplanted s.c into Jan:NMRI:nu/nu (Janvier, France), female mice, 6-8 weeks old and the therapy was started from day 12 when the mean tumour volume reached 165±25 mm³. The drug and the vehicle (2.5% DMSO/5% EtOH/20% PEG200/72.5% saline) were administered intraperitoneally (i.p.) every other day (qod) for a total of 10 injections.

SND164 significantly inhibited tumour growth as shown by the T/C value at day 17 after tumour transplantation (Table 17). There were no death during the study and the treatment was well tolerated with no significant body weight loss.

TABLE 17
Optimal T/C values against glioblastoma U87MG CDX

| | No | | Dose | Optimum T/C, |
|---|---|---|---|---|
| Group | mice | Treatment | (mg/kg/inj) | day 26 |
| A | 10 | vehicle | | |
| B | 10 | SND164 | 10 | 54*** |

*P < 0.05,
** P < 0.01;
***P < 0.001 compared to group A by Two-way-ANOVA

It will be understood that the present invention has been described above by way of example only. The examples are not intended to limit the scope of the invention. Various modifications and embodiments can be made without departing from the scope and spirit of the invention, which is defined by the following claims only.

1. A compound of formula (1): 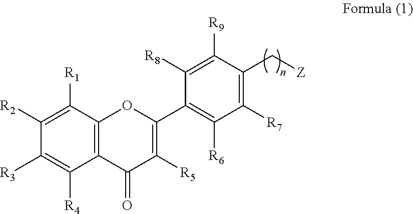
wherein:
Z is selected from: —NR¹¹R¹²;
—N(R¹⁰)—(CH₂)ₚ—NR¹¹R¹²;
—N(R¹⁰)—(CH₂)_{q}—N(R¹⁰)—(CH₂)_{q}—NR¹¹R¹²; and
R¹ and R², independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl , —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl, or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-6;
each p is independently an integer selected from 1 to 4; and
each q is independently an integer selected from 1 to 4.

2. A compound as claimed in claim 1, wherein R¹ and R², independently, are selected from —OH, and —O—C₁₋₄ alkyl.

3. A compound as claimed in claim 2, wherein R¹ and R², are independently selected from —OH and —OCH₃.

4. A compound as claimed in claim 1, wherein R¹ and R², together form —O—CH₂—O—.

5. A compound as claimed in any one or more of the preceding claim, wherein R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}.

6. A compound as claimed in claim 5, wherein R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}.

7. A compound as claimed in claim 6, wherein R³, R⁴, R⁵, R⁶, R⁷, R⁸, and R⁹, independently, are selected from H; halo; —CN; —NO₂; and —NH₂.

8. A compound as claimed in claim 7, wherein R³, R⁴, R⁵, R⁶, R⁷, R⁸, and R⁹ are H.

9. A compound as claimed in claim 1; wherein R¹ and R² are independently selected from —OH and —O—C₁₋₄ alkyl, e.g. —OH and —OCH₃; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

10. A compound as claimed in claim 9; wherein R¹, and R², independently, are selected from —OH and —OCH₃; and R³, R⁴, R⁵, R⁶, R⁷, R⁸, and R⁹, independently, are selected from H; halo; —CN; —NO₂; —SH; —SO₂H; and —NH₂.

11. A compound as claimed in any one or more of the preceding claims; wherein n is an integer from 1 to 4 for example 3 or 4.

12. A compound as claimed in any one or more of the preceding claims; wherein n is 1.

13. A compound as claimed in any one or more of the preceding claims; wherein R¹¹ and R¹² are independently selected from H, C₁₋₆ alkyl, and benzyl, where each R¹¹ and R¹² is optionally substituted with 1-2 R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl.

14. A compound as claimed in claim 13; wherein R¹¹ and R¹² together form a 5- or 6-membered heterocycle optionally substituted with 1 or 2 C₁₋₄ alkyl or benzyl.

15. A compound as claimed in claim 14; wherein the 5- or 6-membered heterocycle is morpholine, piperidine, piperazine, or pyrrolidine optionally substituted with C₁₋₄ alkyl or benzyl.

16. A compound as claimed in any one or more of the preceding claims; wherein Z is —NR¹¹R¹² and n is 3 or 4.

17. A compound as claimed in any one or more of claims 1 to 15; wherein Z is —N(R¹⁰)—(CH₂)ₚ—NR¹¹R¹²; p is 1-4; and n is 1-6.

18. A compound as claimed in any one or more of claims 1 to 15; wherein Z is —N(R¹⁰)—(CH₂)_{q}—N(R¹⁰)—(CH₂)_{q}—NR¹¹R¹²; and q is independently selected from 1-4.

19. A compound as claimed in any of the preceding claims, wherein the compound is a compound of Formula (1A): 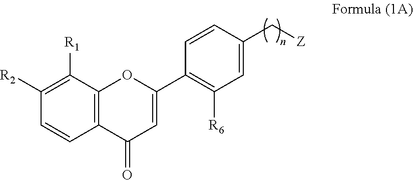
wherein R¹, R², R⁶, n and Z are as defined in claims 1 to 18.

20. A compound as claimed in claim 1, wherein the compound is a compound of Formula (2): 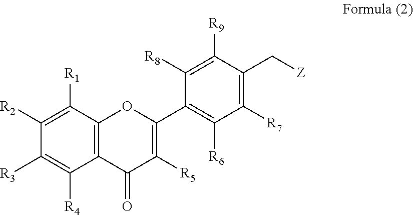
wherein R¹, R², R⁶, n and Z are as defined in claims 1 to 18.

21. A compound as claimed in claim 1, wherein the compound is a compound listed in Table A.

22. A pharmaceutically acceptable salt, multi-salt, solvate or prodrug of a compound as defined in any one of claims 1 to 21.

23. A pharmaceutical composition comprising a compound as defined in any one of claims 1 to 21, or a pharmaceutically acceptable multi-salt, solvate or prodrug as defined in claim 22, and a pharmaceutically acceptable excipient.

24. A compound as defined in any one of claims 1 to 21, or a pharmaceutically acceptable multi-salt, solvate or prodrug as defined in claim 22, or a pharmaceutical composition as defined in claim 23, for use in medicine.

25. A compound as defined in any one of claims 1 to 21, or a pharmaceutically acceptable multi-salt, solvate or prodrug as defined in claim 22, or a pharmaceutical composition as defined in claim 23, for use treating or preventing cancer.

26. A method of treatment or prevention of a disease, disorder or condition, the method comprising the step of administering an effective amount of a compound as defined in any one of claims 1 to 21, or a pharmaceutically acceptable multi-salt, solvate or prodrug as defined in claim 22, or a pharmaceutical composition as defined in claim 23, to thereby treat or prevent the disease, disorder or condition.

27. A method of treatment as claimed in claim 26, wherein the disease, disorder or condition is cancer.
